(19) United States
(12) Patent Application Publication
(10) Pub. No.: US 2011/0313056 A1
(43) Pub. Date: Dec. 22, 2011

(54) GEL FORMATION OF POLYELECTROLYTE AQUEOUS SOLUTIONS BY THERMALLY INDUCED CHANGES IN IONIZATION STATE

(75) Inventors: Michael D. Buschmann, Montreal (CA); Dominic Filion, Montreal (CA); Marc Lavertu, Pointe-Claire (CA)

(73) Assignee: BIO SYNTECH CANADA INC., Laval (CA)

(21) Appl. No.: 13/184,953

(22) Filed: Jul. 18, 2011

Related U.S. Application Data
(63) Continuation of application No. 12/092,484, filed on Aug. 25, 2008, now abandoned.
(60) Provisional application No. 60/733,174, filed on Nov. 4, 2005.

(51) Int. Cl. A61K 47/36

(52) U.S. Cl. 514/777

(57) ABSTRACT

There is disclosed aqueous solutions of polyelectrolytes that can be neutralized by increasing temperature, under suitable polyelectrolyte charge state conditions, in order to obtain a homogeneous gel. This can be achieved by adding an appropriate weak electrolyte to the polyelectrolyte so that an increase of temperature will lead to a spatially homogeneous neutralization of the polyelectrolyte via proton transfer between the polyelectrolyte and the weak electrolyte. The ability of such a system to be thermally sensitive and to gel upon heating relies on the temperature dependence of the ionization equilibrium for the two components. This thermally induced neutralization reduces electrostatic repulsion between polyelectrolyte molecules allowing the manifestation of attractive polyelectrolyte-polyelectrolyte hydrophobic interactions and hydrogen bonding that result in gel formation. These new kinds of thermo sensitive gels can be used for biomedical applications.

### CROSS-REFERENCE TO RELATED APPLICATIONS

This application is a continuation of U.S. Ser. No. 12/092,484, filed on Aug. 25, 2008 which is a 371 National Stage of International Application No. PCT/CA2006/001814, filed on Nov. 6, 2006, which claims the benefit under 35 U.S.C. §119(e) of U.S. provisional application Ser. No. 60/733,174 filed Nov. 4, 2005, the entire contents of which are incorporated herein by reference.

### TECHNICAL FIELD OF THE INVENTION

The present invention concerns thermo-sensitive, charge-state dependant, formation of polyelectrolyte gels.

### BACKGROUND OF THE INVENTION

Chitosan is a polysaccharide obtained by partial deacetylation of chitin (Hoppe-Seyler, Berichte; 3329-3331, 1894). Chitin is insoluble in water while chitosan is soluble when free amino groups of chitosan are sufficiently protonated. Chitosan is inexpensive and commercially available in varying deacetylation ratio (f_{D}). The use of gels based on chitosan and its derivatives for cell and drug delivery has been widely studied (Lavertu et al., J Control Release, submitted 2005; Liu et al., Bioconjugate Chem 14: 782-789, 2003; MacLaughlin et al., J Control Release 56: 259-272, 1998).

Thermo sensitive aqueous solutions based on a chitosan/glycerol 2-phosphate (GP) have been described previously (U.S. Pat. No. 6,344,488). In the system described in U.S. Pat. No. 6,344,488, the glycerol 2-phosphate, which is present partly in an anionic divalent form, was proposed to increase the strength of hydrophobic interactions between chitosan upon heating, thereby forming a thermo-sensitive gel. The phosphate groups were not thought be a direct ionic cross-linking agent of chitosan, as is the role of calcium in calcium alginate systems, due to stearic hindrance. That is to say that an ionic bridge of divalent phosphate linking two charged monovalent amine groups of chitosan is unlikely due to stearic hindrance given the molecular sizes of the molecules involved. Moreover, U.S. Pat. No. 6,344,488 teaches that the gelation is specifically induced by organic mono-phosphate dibasic salts of polyols or sugars. According to this invention the critical feature of this kind of system is the structuring action of the polyol or the sugar part of the organic salt on water that induces chitosan-chitosan hydrophobic interactions via a dehydration effect. The structuring action of the polyol moieties on water thereby reduces the chitosan-water interactions and enhances the chitosan-chitosan interactions. The nontrivial aspect of such a gelation originates essentially from the later polyol-water induced chitosan hydrophobic attractions, which are enhanced upon increasing temperature (temperature-controlled gelation).

International publication WO03/042250 provides a new composition and method for chemically modifying chitosan, including N-substituting or N-cross-linking, under homogeneous conditions by providing neutral aqueous chitosan solutions with enhanced reactivity. The method comprises the steps of preparing a clear aqueous solution of chitosan and of dissolving homogeneously at least one reagent into the solution. The solution of chitosan had to be composed of 0.1 to 10% by weight of a chitosan, and of 0.1 to 20% by weight of at least one buffering agent having a pKa between 6.0 and 7.6. The solution also had to have a pH ranging from 6.8 to 7.2. The reagent to be dissolved in the chitosan solution had to be at a concentration from 0.01 to 10% by weight, and it had to be reactive toward the amine groups of chitosan. This publication therefore teaches the making of an aqueous chitosan solution that is chemically modified or cross-linked by a selective substitution on the amino group of chitosan, and that can be used in the making of a chitosan hydrogel.

The international publication WO01/36000 is teaching a biopolymeric liquid aqueous composition for producing self-gelling systems and gels and a method for preparing such a composition. The composition is comprising an acidic water-based medium, 0.1 to 10% by weight of a pH-gelling acid-soluble biopolymer, and 0.1 to 10% by weight of a water-soluble molecule having a basic character and a pKa between 6.0 and 8.4. The liquid composition has a final pH ranging from 5.8 and 7.4, and forms a stable solid and homogeneous gel within a temperature range from 10 to 70° C. Cosmetic, pharmacological and medical uses of this composition are also presented by this reference.

Aebischer et al. have shown that a core matrix of chitosan can be formed by precipitation induced via neutralization of the amino groups of the polymer (U.S. Pat. No. 6,140,089). In this patent a partly neutralized solution of chitosan containing cells is encapsulated in a permeable or a semi-permeable membrane and then washed several times with physiological saline to allow further neutralization and full precipitation to occur. It is clear here that use of an encapsulating membrane is necessary for this type of dialysis neutralization process. Also, according to Aebischer, the use of dibasic phosphate or any other multivalent anions is not suitable since they will lead to undesirable levels of ionic cross-linking. Aebischer further mentions that if phosphate buffers are used, they should be monobasic. No mention of thermo-sensitivity is made in Aebischer et al.

It should be noted that in the current state of the art, the use of a thermally gelling chitosan solution that is free of organic (polyol) salts, of ionic cross-linking and of encapsulation membranes has not been reported.

### SUMMARY OF THE INVENTION

It is reported herein for the first time the complete mechanism of gelation of thermally gelling chitosan gel composition, which mechanism has now been uncovered and allows for generalization.

The present invention provides a new thermally gelling chitosan gel composition where the mechanism of gelation is based upon changes in ionization state of solution components upon heating, allowing the polyelectrolyte component to form a precipitated network, or hydrogel. One example of such a system is heat induced proton transfer from the cationic polyelectrolyte chitosan to an inorganic phosphate base. This system is free of organic salts, chemical or ionic cross link and encapsulation membranes. It can be used for encapsulation of living cells or their delivery, as well as for drug delivery, protein delivery and gene delivery applications. This new material can be injected into body sites in the liquid state and gels in situ at body temperature and at physiological pH. Several additional systems can be devised using the principles disclosed in this invention, where proper combinations of polyelectrolytes and weak electrolytes will result in changes in ionization state upon heating and thereby produce thermosensitive gels.

### BRIEF DESCRIPTION OF THE DRAWINGS

FIG. 1 shows the rheological behavior upon heating of a chitosan/phosphate solution;

FIG. 2 shows custom experimental apparatus that performs temperature-controlled titrations, while recording temperature, pH and relative light transmittance (L_{T}) of chitosan solutions. The temperature of the solution is controlled via the circulating bath and a titrator adds 0.01M NaOH to the solution. A photodetector assesses laser light transmittance through the beaker and solution to detect phase separation.

FIG. 3 shows the relative light transmittance (L_{T}) along with volume of added titrant V_{T}, both recorded as a function of time, to illustrate the sharp decrease in L_{T} (circle) occurring at a volume of 3 mL injected titrant, in this case. The αₚₛ value is calculated from equation 87, using the Na⁺, Cl⁻ and cₚ concentrations at the corresponding injection volume, neglecting the proton concentration.

FIG. 4 shows the influence of chitosan degree of ionization on pKₐₚ and relative light transmittance under different (4A) temperature T, (4B) ionic strength I_{c} and (4C) fraction of deacetylation f_{D}. Dark symbols represent experimental data obtained in the single phase region (mean±SD; n=3), while lighter grey symbols are data obtained after phase separation. Solid lines are the Poisson-Boltzmann (PB) model fit to data (equations 76, 81, 84 and 87) in the single phase region while dashed lines are the continuation of this model fit into the phase separated region. Finely dotted lines are used to link normalized light transmittance (L_{T}^{N}) to show the occurrence of phase separation where L_{T}^{N} starts to decrease.

FIG. 5 shows the pKₐ or pKₐₚ variation with temperature (equation 14 with reference temperature 5° C.) for glycerol 2-phosphate, inorganic phosphate, D(+)-glucosamine, and chitosan with f_{D}=1.00 in I_{c}=0 obtained from temperature ramp tests. Both D(+)-glucosamine and chitosan experience a significant decrease in pKₐₚ upon heating while the pKₐₚ of inorganic phosphate and glycerol 2-phosphate remained almost constants. Solutions were prepared as described where chitosan had f_{D}=1.00 without added salt. The chitosan solution used in the ramp test (up triangles) had 1.5 mL of NaOH 0.01 N added to achieve α=0.75.

FIG. 6 shows ³¹P chemical shifts of GP solution along with the pH at a given temperature (squares at 5° C., circles at 15° C., up triangles at 25° C. and down triangles at 37° C.) to determine δₐ and δ_{b} values.

FIG. 7 shows the degree of ionization of chitosan versus temperature, measured from GP ³¹P chemical shifts, for various chitosan/GP mixtures (Table 5).

FIG. 8 shows the determination of chitosan precipitation (phase separation) using normalized light transmittance (L_{T}^{N}) value (8A) along with the corresponding pH (8B) to show the decreasing ionization degree of chitosan, indirectly. The simultaneous measurements of L_{T}^{N} and pH were done with the sample mixtures M2 and M3 (see Table 5).

FIG. 9 shows the concentration profile increase of the GP in the DMEM bath solution on top of the gel over time.

FIG. 10 shows different concentration profiles of GP in the gel and the DMEM where x indicates position in the Petri dish from bottom (x=0 mm) to top (x=0.95 mm). Time (in minutes) is indicated next to the corresponding profile.

FIG. 11 shows the orientation of the x-axis in the gel and the washing solution with the bottom of the dish defined as x=0.

FIG. 12 shows a four monomer segment of chitosan (12A) represented with two protonated monomers, a neutral monomer, and an unprotonatable N-acetyl-glucosamine monomer. Each monomer has a length l. FIG. 12B illustrates a smaller cylinder with radius a, corresponding to the chitosan molecule that is contained in its electrolyte envelope extending to radius b. Representative profiles of electrostatic potential ψ(r), weak electrolyte concentration, c₋, and co-ion concentration, c₊, are shown for the case of I_{c}=15 mM NaCl at α=0.75 and f_{D}=1.00. The circle indicates the electrostatic potential at the surface of the polyelectrolyte, ψ|ᵣ₌ₐ.

FIG. 13 shows ramp temperature experiment on a chitosan-phosphate solution (phosphate/glucosamine molar ratio of 1.67). The precipitation is shown by a decrease in the transmittance and a decrease in pH that coincide at ˜42° C.

FIG. 14 shows temperature ramp experiment of two chitosan-GP solutions with GP/glucosamine molar ratios of 3.67 and 5.

FIG. 15 shows ramp temperature experiment on a solution of chitosan-GP (molar ratio GP/glucosamine=3.67) and a solution of chitosan-disodium phosphate (molar ratio phosphate/glucosamine=1.67). The phosphate solution has a higher initial pH because of its higher pKₐ (7.11 vs 7.00 at 25° C.).

FIG. 16 shows ramp temperature experiment on a chitosan-MES solution (MES/glucosamine molar ratio of 5). The precipitation is shown by a decrease in the transmittance.

### DETAILED DESCRIPTION OF THE PREFERRED EMBODIMENT

The following description will be made by considering this chitosan/dibasic sodium phosphate or inorganic phosphate system but one should keep in mind that the mechanism of gelation of this system can be extended to virtually any polyelectrolyte aqueous system that presents specific characteristics to be described herein.

An aqueous chitosan solution at physiological pH that gels upon heating is described in accordance with the present invention. A method for preparing the gel is presented wherein a chitosan/dibasic sodium phosphate mix is heated from room temperature (approximately 20° C.) to body temperature (approximately 37° C.). The mechanism of formation of the gel is described in terms of a heat-induced proton transfer from chitosan to dibasic sodium phosphate resulting in chitosan neutralization and homogeneous precipitation or gel formation. The temperature of gelation can be adjusted by changing phosphate/glucosamine ratios.

The present invention is based on the discovery that chitosan can be homogeneously neutralized by heating in order to form a gel. The characterization of its physico-chemical properties are described in the section “Detailed description of the characterization of the polyelectrolyte and the weak electrolyte”. The mechanism of gel formation by heating of a chitosan/dibasic sodium phosphate gel is presented herein.

One embodiment of the present invention provides a thermally sensitive polyelectrolyte composition comprising a solution of a polyelectrolyte; and a weak electrolyte, said weak electrolyte being dissolved in the solution of polyelectrolyte and causing said polyelectrolyte to precipitate and form a gel upon heating, when said composition components reach specific charge state values.

Another embodiment of the invention provides a method for preparing a thermally sensitive polyelectrolyte composition comprising a solution of a polyelectrolyte; and a weak electrolyte, said method comprising the step of dissolving at a temperature below the gelling temperature of the composition a weak electrolyte in the solution of polyelectrolyte without causing gelation of the composition to occur, said composition turns into a gel upon heating when said composition components reach specific charge state values.

In one embodiment of the present invention, the first step in the preparation of a solution that forms a gel is to partially neutralize the polyelectrolyte chitosan and bring it close to precipitation via addition of a weak base such as dibasic sodium phosphate. The exact level of neutralization required depends on parameters such as chitosan concentration, its degree of deacetylation, acetyl group distribution, and its molecular weight, as well as the ionic strength of the solution and temperature. After this partial neutralization step, the solution is then heated. At this point, since the tendency of chitosan to release its protons with increasing temperature is significantly greater than that of the dibasic sodium phosphate (the dissociation constant of chitosan increases with temperature while this parameter is quite stable for the dibasic sodium phosphate providing a proton sink), there is a transfer of protons from chitosan to the dibasic sodium phosphate. Thus, in this example, our discovery consists mainly in the demonstration that heating of the solution induces a homogeneous proton transfer from chitosan to dibasic sodium phosphate resulting in homogeneous precipitation of the polysaccharide. In order to have an appreciable neutralization of chitosan, there must be enough dibasic sodium phosphate to accept these protons. Under these conditions, the transfer of protons is sufficient to bring the polymer to precipitation and induce the sol-gel transition. The gel formation is in fact a block precipitation of the polymer resulting from a homogeneous neutralization of the polyelectrolyte induced by heating. This neutralization allows attractive hydrophobic interactions between the chitosan chains that will come together and form a three-dimensional network.

The dibasic sodium phosphate acts as a proton sink that allows deprotonation of the chitosan during heating. There is therefore no ionic cross-link between the divalent anionic phosphate and the chitosan so that the former is free to diffuse out of the gel. The proof that the sodium phosphate do not form any cross-link with chitosan is described in the section “Detailed description of the proof of absence of cross-links between the polyelectrolyte and the weak electrolyte”.

To determine the condition for which a reduction of the ionization degree of chitosan occurs upon heating, a set of equations was used to solve weak electrolyte systems, namely dissociation equations, species conservation and electroneutrality.

It should be noted that the dissociation of protons occurs at glucosamine monomers of chitosan. Therefore, the condition is established using equations related to this monomer. When using GlcNH₂, we refer to the neutral form of chitosan monomer and when using GlcNH₃⁺ we refer to its ionized form. When using glucosamine (Glc) alone, we refer to all monomers including the neutral and ionized form. The same applies for phosphate using PO₄³⁻ for its trivalent form, PO₄²⁻ for its divalent form, PO₄ for its monovalent form, PO₄ for its neutral form and P by itself refers to all phosphate ions. The system is restricted to pH between 5 and 8 and to monomer concentrations over 1 mM. It should be noted that those restrictions are fully satisfied for the present embodiment and that they are needed to make approximations that facilitate calculations.

The dissociation equations of glucosamine and phosphate are

$$\begin{matrix}{K}_{ap}^{Glc} = \frac{{c}_{{H}^{+}} {c}_{{GlcNH}_{2}}}{{c}_{{GlcNH}_{3}^{+}}} & ( 1 ) \\ {K}_{a 1}^{P} = \frac{{c}_{{H}^{+}} {c}_{{PO}_{4}^{-}}}{{c}_{{PO}_{4}}} & ( 2 ) \\ {K}_{a 2}^{P} = \frac{{c}_{{H}^{+}} {c}_{{PO}_{4}^{2 -}}}{{c}_{{PO}_{4}^{-}}} & ( 3 ) \\ {K}_{a 3}^{P} = \frac{{c}_{{H}^{+}} {c}_{{PO}_{4}^{3 -}}}{{c}_{{PO}_{4}^{2 -}}} & ( 4 )\end{matrix}$$

The constant Kₐₚ^{Glc} is the object of the following section whereas values for Kₐ₁^{P}, Kₐ₂^{P} and Kₐ₃^{P} can be found in the literature (Voet and Voet, Biochimie 2^{e} edition, De Boeck, Italia, John Wiley & Sons, 1361 p., 1998) to be approximately 2.2, 6.8 and 12.4 respectively. When using the value for Kₐ₁^{P} and the restricted pH range of 5 to 8, we have

$$\frac{{K}_{a 1}^{P}}{{c}_{{H}^{+}}} = \frac{{c}_{{PO}_{4}^{-}}}{{c}_{{PO}_{4}}} \geq \frac{{10}^{- 2.2}}{{10}^{- 5}} = 630 \qquad ( 5 )$$

When using the value for Kₐ₃^{P} and in the restricted pH range, we have

$$\frac{{c}_{{H}^{+}}}{{K}_{a 3}^{P}} = \frac{{c}_{{PO}_{4}^{2 -}}}{{c}_{{PO}_{4}^{3 -}}} \geq \frac{{10}^{- 8}}{{10}^{- 12.4}} = 25120 \qquad ( 6 )$$

Therefore, PO₄ and PO₄³⁻ concentrations can be neglected leading to the conservation equation

c_{P}≅c_{PO}_{₄}_{^{−}}+c_{PO}_{₄}_{^{2−}} (7)

Conservation of Glc monomers gives

c_{Glc}=c_{GlcNH}_{₂}+c_{GlcNH}_{₃}_{⁺} (8)

The requirement of electroneutrality is written

c_{Na}_{⁺}+c_{GlcNH}_{₃}_{⁺}−c_{Cl}_{^{−}}−c_{PO}_{₄}_{^{−}}−2c_{PO}_{₄}_{^{2−}}=0 (9)

since H⁺ and OH⁻ concentrations may be neglected when pH is between 5 and 8 and concentrations of the species cited in equation 9 are greater than 10⁻³ M.

Having α defined as the ionized ratio of the glucosamine monomer

$$α = \frac{{c}_{{GlcNH}_{3}^{+}}}{{c}_{Glc}} \qquad ( 10 )$$

The equation system is normalized by defining χ, β and γ as

$$χ = \frac{{c}_{{PO}_{4}^{2 -}}}{{c}_{P}} , \\ β = \frac{{c}_{P}}{{c}_{Glc}} \\ and \\ γ = \frac{{c}_{{Na}^{+}} - {c}_{{Cl}^{-}}}{{c}_{Glc}} \qquad ( 11 )$$

where c_{Na+} and c_{Cl-} represent the concentration of the dissociate ions Na⁺ and Cl⁻, respectively.

Using these normalized parameters, electroneutrality (equation 9) can be expressed as

α+γ=β(1+χ) (12)

Temperature induced gelation for the chitosan phosphate system will occur if chitosan charge state is sufficiently reduced upon heating to allow precipitation. Thus one necessary condition for inducing thermosensitive gelation via heat-induced neutralization is that

$$\frac{\partial α}{\partial T} < 0.$$

It can be shown (see section “The degree of ionization of polyelectrolytes in solution varies with temperature in a manner predicted by the temperature-dependence of their dissociation constants”) that

$$\frac{\partial α}{\partial T} < 0$$

is satisfied if

$$\frac{\partial {pK}_{ap}^{Glc}}{\partial T} < \frac{\partial {pK}_{a 2}^{P}}{\partial T} \qquad ( 13 )$$

Alternatively, the demonstration that the degree of ionization of polyelectrolytes in solution varies with temperature in a manner predicted by the temperature-dependence of their dissociation constants can be made as follows:

The following demonstration is derived for a chitosan/dibasic sodium phosphate aqueous system. However, it can be generalized to any system composed of two weak electrolytes that are each in a single dissociation equilibrium (as for phosphate in the range of pH 5 to 8 where the two other dissociation equilibriums can be neglected). The theoretical expression of the apparent pKₐ of a polyelectrolyte is given by the following equation (see section “Poisson-Boltzmann cylindrical cell model predicts pKₐₚ variation with the degree of ionization for a polyelectrolyte”)

$${pK}_{ap} ( T ) = pH ( T ) - {log}_{10} \frac{1 - α}{α} = {pK}_{0} ( T ) - \frac{e ψ {\vert}_{r = a}}{ln 10 kT}$$

For weak polyelectrolytes, the pKₐₚ variation with its degree of ionization is generally linear. Titration experiments on chitosan show that this also applies to chitosan (see Table 2), so that the pKₐₚ of chitosan can be expressed as

$${pK}_{ap} ( T ) \approx {pK}_{0} ( T ) - m ( T ) α with m ( T ) α \approx \frac{e ψ {\vert}_{r = a}}{ln 10 kT} \qquad ( 1 a )$$

Theoretical calculations and experiment on chitosan show that m(T) doesn't vary significantly with temperature. The expression of the pKₐₚ is rewritten taking m as a constant:

pKₐₚ(T)≈pK₀(T)−mα (2a)

For chitosan, m is positive and the pKₐₚ decreases as the charge state α increases. The dissociation equations 1 and 3 are rewritten using α, χ and equation 2a:

$$\begin{matrix}\begin{matrix}{K}_{ap}^{Glc} = \frac{{c}_{{H}^{+}} ( 1 - α )}{α} ⇒ ln ( {10}^{- {pK}_{ap}} ) \\ = ln ( 1 - α ) - ln α + ln {c}_{{H}^{+}} \\ \approx ln ( {10}^{m α} {K}_{0}^{Glc} )\end{matrix} & ( 3 a ) \\ \begin{matrix}{K}_{a 2}^{P} = \frac{{c}_{{H}^{+}} χ}{1 - χ} ⇒ ln {K}_{a 2}^{P} \\ = - ln ( 1 - χ ) + ln χ + ln {c}_{{H}^{+}}\end{matrix} & ( 4 a )\end{matrix}$$

The normalized form of electroneutrality is given by equation 9:

$$α + γ = β ( 1 + χ ) ⇒ χ = \frac{α + γ - β}{β} \qquad ( 5 a )$$

Knowing that β and γ are invariant, the total differential of equations 3a, 4a and 5a are (the almost equal sign ≈ is replaced by equality sign for convenience in equation 3a):

$$\begin{matrix}d ln {K}_{0}^{Glc} = - \frac{d α}{1 - α} - \frac{d α}{α} - m ln 10 d α + \frac{d {c}_{{H}^{+}}}{{c}_{{H}^{+}}} & ( 6 a ) \\ d ln {K}_{a 2}^{P} = \frac{d χ}{1 - χ} + \frac{d χ}{χ} + \frac{d {c}_{{H}^{+}}}{{c}_{{H}^{+}}} & ( 7 a ) \\ d χ = \frac{d α}{β} & ( 8 a )\end{matrix}$$

By subtracting equation 7a from equation 6a, we obtain:

$$d ln {K}_{0}^{Glc} - d ln {K}_{a 2}^{P} = - \frac{d α}{1 - α} - \frac{d α}{α} - m ln 10 d α - \frac{d χ}{1 - χ} - \frac{d χ}{χ} \qquad ( 9 a )$$

Using the expression of dχ given by equation 8a, equation 9a is rewritten:

$$d ln {K}_{0}^{Glc} - d ln {K}_{a 2}^{P} = - ( \begin{matrix}\frac{1}{1 - α} + \frac{1}{α} + m ln 10 + \\ \frac{1}{β ( 1 - χ )} + \frac{1}{βχ}\end{matrix} ) d α \qquad ( 10 a )$$

and by using

$$d pK = d ( - {log}_{10} K ) = - \frac{1}{ln 10} d ln K$$

and by dividing by dT, we obtain

$$\frac{d α}{d T} = \frac{ln 10 ( \frac{d {pK}_{0}^{Glc}}{d T} - \frac{d {pK}_{a 2}^{P}}{d T} )}{( \frac{1}{α ( 1 - α )} + \frac{1}{βχ ( 1 - χ )} + m ln 10 )} \qquad ( 11 a )$$

Thus, since α and χ range from 0 to 1 and that β and m are positive,

$$\frac{d α}{d T} < 0$$

$$if \frac{d {pK}_{0}^{Glc}}{d T} < \frac{d {pK}_{a 2}^{P}}{d T} .$$

Note that m is positive for a cationic polyelectrolyte. Note also that for simple acid/base electrolytes, the variation of α with temperature is obtained from equation 11a taking m=0. Equation 11a predicts an important change in charge state when

$$\vert \frac{d {pK}_{0}^{Glc}}{d T} - \frac{d {pK}_{a 2}^{P}}{d T} \vert and β = \frac{{c}_{P}}{{c}_{Glc}}$$

have high values and when α or χ are not too close from 0 or 1 (this last condition can be satisfied if the pKₐ values of the two electrolytes are similar)

The above derivations can now be extended in an obvious manner to polyanion/cationic weak electrolyte systems

The preferred way to characterize the pKₐₚ (or pKₐ) variation with temperature described in equation 13 is described in the section “Detailed description of the characterization of the polyelectrolyte and the weak electrolyte”.

FIG. 1 is a rheological measurement of a chitosan mixed with sodium phosphate as described in Example 1. The rheological measurements were performed on a Bohlin rheometer (Model CVO50) with a C40 rod at 1 Hz in a manner similar to that described in Chenite et al. (Chenite et al., Carbohyd Polym 46: 39-47, 2001) with a rate of increase in temperature of 1° C./min. The measurement clearly shows the sol-gel transition occurring near 37° C.

Polyelectrolytes and weak electrolytes that can be used to obtain thermally sensitive gels in accordance with one embodiment of the invention are as described in Table 1 below.

TABLE 1
List of polyanions, polycations, counter anions and counter cations.
Polyanions and Polycations

| | |
|---|---|
| Alginate | Polyimide |
| Glycosaminoglycans | Polylysine |
| Hyaluronate | Polysaloxine |
| Polyacrylic acid | Synthetic homo and block copolymers |
| Polyaniline sulfonate | containing carboxylic, amino, sulfonic, |
| Polyascorbate | phosphonic, phosphenic functionalities |
| Polyaspartate | with or without other functionalities such |
| Polyglutamate | as hydroxyl, thiol, alkoxy, aryloxy, |
| Polylactic acid | acyloxy, aroyloxy etc. |
| | Polyglycolic acid |

Counter anions

| | |
|---|---|
| Aliphatic, saturated, unsaturated, | Geminal bis phosphonate |
| helicyclic, acyclic, aromatic, heterocyclic, | Vicinal bis phosphonate |
| alkyl and aryl phosphonate & phosphinate | Pyrophosphate |
| Inorganic carbonate | Inorganic phosphate |
| Inorganic sulfate | L-Serine phosphate |
| Methylene bis phosphonate | Polyphosphate |

Counter cations

| | |
|---|---|
| Adenosine | Aliphatic, acyclic, alicyclic, heterocyclic, |
| Thymidine | mono-, di- and tri-substituted amines |
| Arginine | Ethylene diamine |
| Galactosamine | Glucosamine |
| Guanidine | Imidazol |
| Lysine | Substituted and un-substituted aryl |
| | amines |

### Detailed Description of the Characterization of the Polyelectrolyte and the Weak Electrolyte

The property to characterize is the variation of the dissociation constant pKₐₚ with temperature of the polyelectrolyte and of the weak electrolyte. When this property is determined, we can predict if a proton transfer will occur when the temperature is varied and consequently predict system components and compositions that form thermogelling systems.

The characterization of pKₐₚ may be executed by measuring the pH variation when the temperature is varied. In order to test temperature-induced changes in pKₐₚ, we use the relationship

$$\begin{matrix}\frac{d {pK}_{ap}}{d pH} ≅ 1 & if ( {c}_{{H}^{+}} + {c}_{{OH}^{-}} )\end{matrix} ( \frac{1}{{c}_{{HA}^{+}}} + \frac{1}{{c}_{A}} ) << 1 \qquad ( 14 )$$

where we consider the case of a cationic group on the polyelectrolyte (or electrolyte) (HA⁺ [symbol] H⁺+A) in the presence of a strong acid or base (see section “Derivation of dpKₐₚ/dpH≈1” for proof of equation 14). Thus, the variation in pKₐₚ (or pKₐ) with temperature is assessed by measuring changes in pH as long as the polyelectrolyte (or electrolyte) is not totally in dissociated or associated form and if the proton and hydroxyl ion concentrations are low compared to the polyelectrolyte monomer (or electrolyte) concentration. For our example system, chitosan is only soluble for acidic pH, these conditions are satisfied when the pH>˜4 or equivalently when α<˜0.95. Then the temperature induced change in pKₐₚ with respect to that of a reference temperature, ΔpKₐₚ(T), can be determined from the corresponding pH difference via

ΔpKₐₚ(T)=pKₐₚ(T)−pKₐₚ(T_{ref})≅pH(T)−pH(T_{ref}) (15)

where T_{ref} is an arbitrary reference temperature (such as 25° C.).

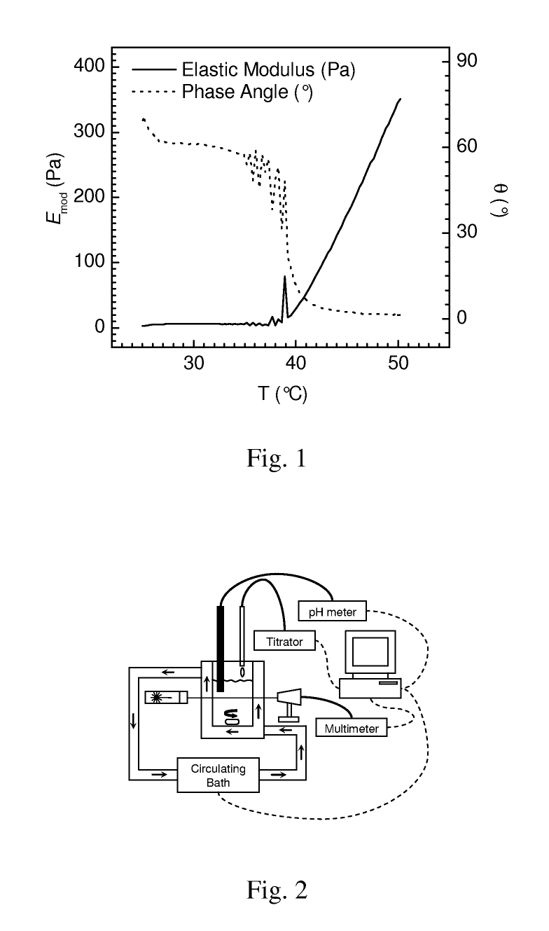

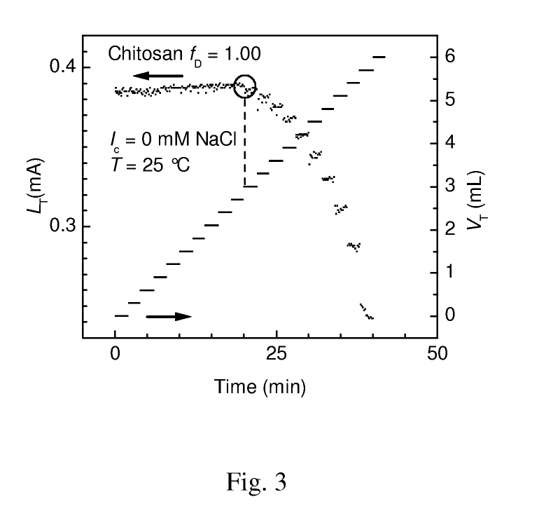

An experimental apparatus can be used (FIG. 2) to perform simultaneous titration and laser light relative transmittance (L_{T}) measurements to detect phase separation of chitosan solutions. This apparatus can also be used to characterize the temperature dependence of pKₐ of D(+)-glucosamine (see Neuberger and Fletcher 1971 for similar results), inorganic phosphate and glycerol 2-phosphate (see Fukada and Takahashi, Proteins—Structure, Function and Genetics 33: 159-166, 1998 for similar results) by measuring pH during temperature ramp tests while respecting the condition stated in equation 14. Solution temperature is controlled using a 50 mL reaction jacketed beaker (Kontes, Cat. No. 317000-0050) coupled to a circulating bath (Neslab, model RT-111) with continuous stiffing during the titration. The pH electrode is calibrated with NIST standards at the particular temperature of constant temperature tests (5, 20, or 37° C.) and at 5° C. for the temperature ramp test, where the automatic temperature compensation probe corrected for the temperature dependence of the pH electrode. Measurements are performed with one of the following two pH/temperature probes: 1) pH electrode (Accumet, Cat. No. 13-620-287), temperature probe (Accumet, Cat. No. 13-620-16) and pH meter (Accumet, Model 20) or 2) combined pH electrode and temperature probe (Orion, Cat. No. 617500) and pH meter (Orion, Model 555A). The addition of 0.01 N NaOH titrant was controlled by an automatic titrator (Schott, Titronic Universal 20 mL) To detect phase separation, laser light relative transmittance, LT, is measured throughout titration using a 635-nm diode laser beam (Coherent, 5 mW, 31-0128) passing through the solution and walls of the beaker with detection by a photo detector (Coherent, Laser-Q VIS, 33-0241) that produces a current (proportional to light intensity) that is read by a multimeter (Fluke, model 45 Dual display). The point of phase separation was characterized by a sharp decrease of L_{T} following injection of titrant (see FIG. 3). The value of α at which these L_{T} values decreased and indicated phase separation is called αₚₛ. A computer controlled the titration burette and bath temperature in addition to acquiring pH, temperature and L_{T} data.

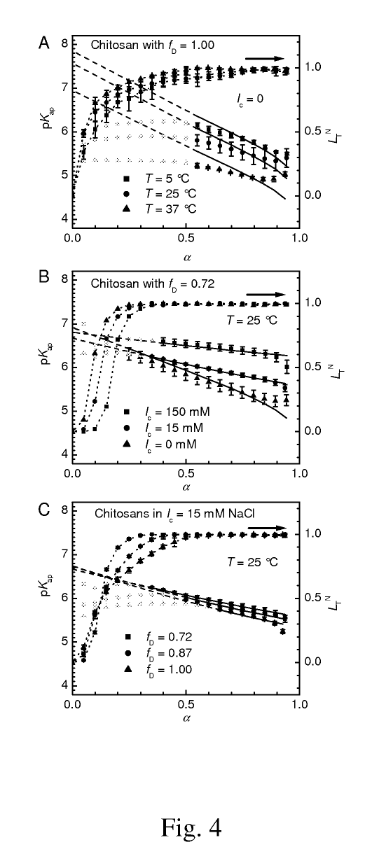

The dissociation constant of the polyelectrolyte may vary for different ionic strengths, for different polyelectrolyte structures (modifying its hydrophobicity or ability to form hydrogen bonds) for different temperatures. Therefore, titration curves can be obtained to measure the dissociation constant and its variation with these parameters. For chitosan, we present pKₐₚ value obtained from titration curves at three different temperatures (FIG. 4A), in three different ionic strengths (0, 15 and 150 mM of NaCl) (FIG. 4B) and using three different chitosans bearing fractions of deacetylated monomeric units (f_{D}) equal to 0.72, 0.87 and 1.00 (FIG. 4C). For each titration condition, the value αₚₛ obtained is presented in Table 2. The pKₐₚ value for a neutralized polyelectrolyte chain (called pK₀) is also shown in Table 2. For weak polyelectrolytes, the pKₐₚ variation with its degree of ionization is generally linear. Therefore, slope values can also be obtained from experimental data and are shown in Table 2.

TABLE 2
Degree of ionization of chitosan at phase separation, αₚₛ, as well
as pK₀ and ΔpKₐₚ/Δα (C_{Ic}), measured at 25° C. for chitosans
with different deacetylation fraction f_{D} and in solutions of
different ionic strength, I_{c}.

| | I_{c} | | f_{D} | |
|---|---|---|---|---|
| | (mM) | 0.72 | 0.87 | 1.00 |
| αₚₛ ^{a} (±0.05) | 0 | 0.25^{b} | 0.35 | 0.50^{b} |
| | 15 | 0.30 | 0.40 | 0.55 |
| | 150 | 0.35 | 0.50 | 0.65 |
| pK₀^{PB c} | 0 | 6.9 ± 0.1 | 7.0 ± 0.1 | 7.5 ± 0.2 |
| | 15 | 6.7 ± 0.1 | 6.7 ± 0.1 | 6.7 ± 0.1 |
| | 150 | 6.8 ± 0.1 | 6.9 ± 0.1 | 6.7 ± 0.1 |
| pK₀^{lin d} | 0 | 6.7 ± 0.1 | 6.9 ± 0.1 | 6.7 ± 0.1 |
| | 15 | 6.6 ± 0.1 | 6.4 ± 0.1 | 6.5 ± 0.1 |
| | 150 | 6.8 ± 0.1 | 6.4 ± 0.1 | 6.2 ± 0.2 |
| −ΔpKₐₚ/Δα^{PB e} | 0 | 2.0 ± 0.2 | 2.3 ± 0.2 | 2.6 ± 0.2 |
| (C_{Ic}^{PB}) | 15 | 1.1 ± 0.1 | 1.3 ± 0.2 | 1.5 ± 0.2 |
| | 150 | 0.6 ± 0.1 | 0.7 ± 0.1 | 0.8 ± 0.1 |
| −ΔpKₐₚ/Δα^{lin f} | 0 | 1.8 ± 0.1 | 1.4 ± 0.1 | 1.4 ± 0.1 |
| (C_{Ic}^{lin}) | 15 | 1.0 ± 0.1 | 0.9 ± 0.1 | 1.2 ± 0.2 |
| | 150 | 0.6 ± 0.1 | 0.6 ± 0.1 | 0.7 ± 0.2 |

^{a}Calculation of αₚₛ from experimental measurements (n = 3 with error of ±0.05 due to measurement accuracy).
^{b}Similar values were obtained at 5° C. and 37° C. with n = 3.
^{c}pK₀^{PB} are values of pK₀ obtained via the PB fit. The error is represented as half the difference of pK₀ obtained with an inner cell radius a set to 0.6 nm versus 1.0 nm
$( error = \frac{{pK}_{0}^{a = 0.6 nm} - {pK}_{0}^{a = 1.0 nm}}{2} ) .$ ^{d}pK₀^{lin} are y-axis intercepts obtained from a linear fit of the pKₐₚ in the non phase separated region and with α ≦ 0.85 (n = 3).
^{e}ΔpKₐₚ/Δα^{PB} is obtained at α = 0.85 using the pK₀ obtained via the PB fit. The error is represented as half the difference obtained with an inner cell radius a set to 0.6 nm versus 1.0 nm
$( error = \frac{{( {pK}_{ap}^{a = 1.0 nm} \vert}_{α = 0.85} - {pK}_{0}^{a = 1.0 nm} ) - {( {pK}_{ap}^{a = 0.6 nm} \vert}_{α = 0.85} - {pK}_{0}^{a = 0.6 nm} )}{2 \cdot 0.85} ) .$ ^{f}ΔpKₐₚ/Δα^{lin} are slope values obtained by a linear fit of the pKₐₚ in the non phase separated region and with α ≦ 0.85 (n = 3).

These three ultrapure chitosans were provided by Bio Syntech (Laval, Qc, Canada) having number average molecular weight (Mₙ) ranging from 65 to 220 kDa and a polydispersity index (PDI=M_{w}/Mₙ) of 1.5 to 1.7. A 1.0 N NaOH (Aldrich, Cat No. 31, 951-1) and 1.0 N HCl (Aldrich, Cat No. 31, 894-9) were used to prepare the titrant solution and to dissolve chitosan, respectively. NaCl (Fisher Scientific, Cat No. S271-1) was used to adjust ionic strength (I_{c}) of chitosan solutions.

The dissociation constant of phosphate, glucosamine monomer and glycerol 2-phosphate also vary with temperature. Therefore, temperature ramp tests were performed by modifying the temperature and measuring the pH of the following solutions. Inorganic phosphate solutions at 50 mM concentration at α=0.5 by mixing equal amount of monobasic phosphate (Sigma, Cat. No. S-5011) and dibasic phosphate (Sigma, Cat. No. S-9713). Monomeric glucosamine (non-polyelectrolyte) solutions were prepared by adding 12.9 mg d(+)-glucosamine hydrochloride (Sigma, Cat. No. G1514) to 20 mL distilled and de-ionized water to obtain 3.00 mM d(+)-glucosamine with 3.00 mM Cl⁻ weak electrolyte. Further addition of 0.3 mL of 0.1 N NaOH solution produces a solution with α≅0.95 that was used for temperature ramp tests described below (equation 14 is satisfied since pH>5.8, and dpKₐ/dpH=1.00±0.01). Glycerol 2-phosphate (GP) solutions at 50 mM concentration with α=0.5 were then prepared by adding 297 mg GP (Sigma, Cat. No. G9891) to 20 mL distilled and de-ionized water followed by addition of 0.5 mL of 1 N HCl (equation 14 condition is satisfied since pH=6.2, and dpKₐ/dpH=1.00±0.01).

In order to prepare chitosan solutions with precise concentration, chitosan powder was dried at 60° C. for 2 days using a heated centrifugal vacuum concentrator (Savant Speedvac, model SS11) and kept in a desiccator until use. Chitosan was dissolved in dilute HCl at a glucosamine monomer to HCl molar ratio of 1:1 so that ionizable sites on the polymer and their weak electrolytes (Cl⁻) were present in equal concentrations in the solution, each at 3 mM. To prepare solutions, dried chitosan was first added to de-ionized water and stirred to disperse the powder prior to adding HCl. The solution was then stirred overnight to ensure complete dissolution of chitosan. The NaCl concentration (I_{c}) of the solution was adjusted by adding appropriate amounts of 5 M NaCl. At the highest level of added salt used in our study, i.e. I_{c}=150 mM, the glucosamine monomer and HCl concentrations were diluted to 2.91 mM.

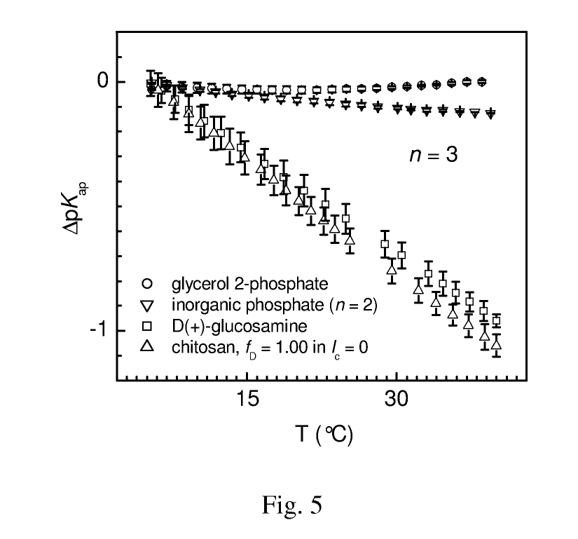

A theoretical cylindrical cell model can be used to solve the Poisson-Boltzmann (PB) equation and fit or predict the experimental data (see section “Poisson-Boltzmann cylindrical cell model predicts pKₐₚ variation with the degree of ionization for a polyelectrolyte” or Marcus, R. A., J Chem Physics 23: 1057-1068, 1955). A useful simplification to the non linear PB model is pKₐₚ=pK₀−C_{T}(T−T_{ref})−C_{Ic}α where we found C_{T}=0.03/° C. (FIG. 4A and FIG. 5) to be independent of I_{c} and f_{D} while C_{Ic}(I_{c}, f_{D}) and pK₀(T_{ref}, I_{c}, f_{D}) do depend on I_{c} and f_{D} and are shown in at T_{ref}=25° C.

In order to measure the variation of the polyelectrolyte ionization degree (chitosan in the present case), we performed NMR measurements of the glycerol 2-phosphate (GP) ³¹P chemical shift. Glycerol 2-phosphate titration curve at different temperatures allow determination pKₐ₂^{GP}, δₐ and δ_{b} by fitting pH and δ values of the following equation

$$pH = {pK}_{a 2}^{GP} + log ( \frac{δ - {δ}_{a}}{{δ}_{b} - δ} ) \qquad ( 16 )$$

where δₐ and δ_{b} are the chemical shifts of ³¹P in the monovalent and divalent form of GP, respectively.

This last equation is valid for ideal solutions where the proton activity coefficient is γ_{H}_{⁺}=1.

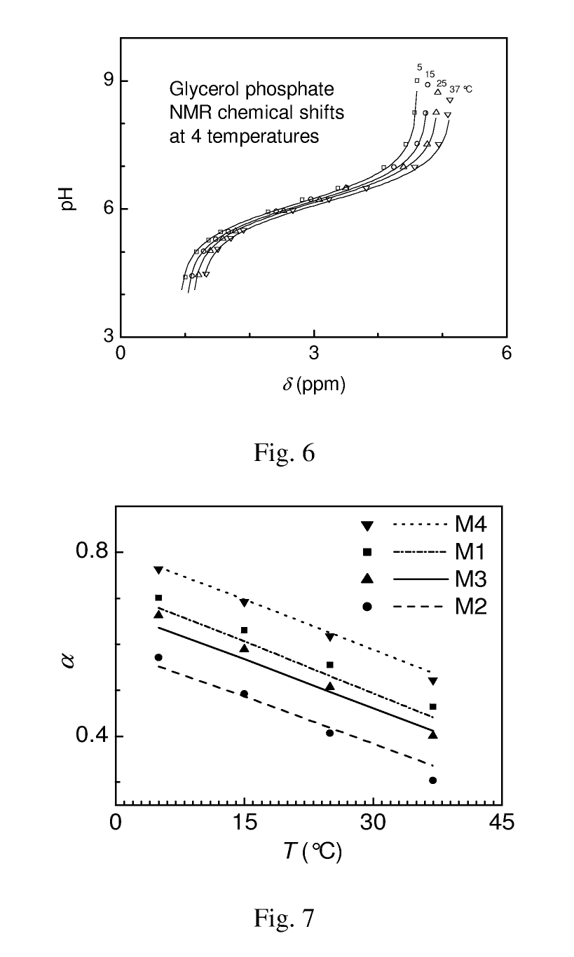

We dissolve 0.594 g of glycerol 2-phosphate (Sigma, No. Cat. G-9891, 297 g/mol with 4.5 moles H₂O per mole of GP) in 18 mL H₂Odd and 2 mL D₂O (Aldrich, No. Cat. 15,188-2-250G) in a graduate cylinder giving a C_{GP} concentration of 100 mM. A total of 11 solutions (see Table 3) with different ionization degree are prepared from this base solution. The pH is measured with an Accumet meter, model 20 using an electrode 9803BN from Orion. Titrations data along with fitted curves are shown in FIG. 6. Table 4 shows the values obtain for pKₐ₂^{GP}, δₐ and δ_{b}.

TABLE 3
Titration of glycerol 2-phosphate with the addition of 1.008M HCl.

| | V_{HCl} (1.008M HCl) | V_{T} | c_{Cl}_{^{−}} | c_{GP} | χ_{GP} |
|---|---|---|---|---|---|
| Sample | μL | mL | mM | | |
| 1a | 0 | 20.00 | 0.00 | 100 | 1.00 |
| 1b | 18.35 | 18.52 | 1.00 | 99.90 | 0.99 |
| 1c | 67.47 | 17.09 | 4.98 | 99.51 | 0.95 |
| 1d | 153.9 | 15.74 | 14.78 | 98.53 | 0.85 |
| 1e | 278.4 | 14.52 | 33.83 | 96.64 | 0.65 |
| 1f | 187.2 | 13.21 | 47.64 | 95.27 | 0.50 |
| 1g | 166.0 | 11.87 | 61.07 | 93.94 | 0.35 |
| 1h | 193.3 | 10.56 | 78.39 | 92.22 | 0.15 |
| 1i | 41.47 | 9.11 | 82.63 | 91.80 | 0.10 |
| 1j | 34.64 | 7.64 | 86.82 | 91.39 | 0.05 |
| 1k | 27.84 | 6.17 | 90.98 | 90.97 | 0.00 |

In order to obtain a variation of the degree of the second ionization of GP, we diluted the volume V_{T} of the GP solution with the volume V_{HCl} of 1.008M HCl solution leading to a c_{Cl} Cl⁻ concentration and c_{GP} GP concentration.

TABLE 4
Values for δₐ and δ_{b} determined from fitting
of experimental data following equation 16.

| T | δₐ | δ_{b} | |
|---|---|---|---|
| ° C. | ppm | ppm | pKₐ₂^{GP} |
| 5 | 0.9154 | 4.6089 | 6.13 |
| 15 | 1.0202 | 4.7769 | 6.14 |
| 25 | 1.1166 | 4.9410 | 6.16 |
| 37 | 1.2272 | 5.1464 | 6.16 |

We then use the pKₐ₂^{GP}, δₐ and δ_{b} values found at different temperatures to determine χ with the following equation

$$χ = \frac{δ - {δ}_{a}}{{δ}_{b} - {δ}_{a}} \qquad ( 17 )$$

In order to measure the variation of the degree of ionization of chitosan with temperature when mixed with GP, we measured the GP ³¹P chemical shift at different temperatures. Table 5 presents the masses used to prepare 4 chitosan/GP mixtures in order to obtain different values of β and γ. The preparation is described herein.

TABLE 5
Chitosan and glycerol 2-phosphate solutions mass before the 1:1
combination. The mass mc of chitosan was dissolved with the HCl
volume VHCl in order to obtain a final concentration of glucosamine
monomer c_{c}^{g-f} and the mass m_{GP} of GP was used to
obtain a final concentration of c_{GP}^{f}

| | m_{c} | c_{c}^{g-f} | V_{HCl} | c_{HCl}^{f} | m_{GP} | c_{GP}^{f} | | |
|---|---|---|---|---|---|---|---|---|
| Sample mixture | mg | mM | mL | mM | mg | mM | β* | γ* |
| M1 | 333.05 | 30 | 1.446 | 30 | 0.4455 | 30 | 1.0 | 1.0 |
| M2 | 333.05 | 30 | 1.157 | 24 | 0.4455 | 30 | 1.0 | 1.2 |
| M3 | 333.05 | 30 | 1.446 | 30 | 0.5346 | 36 | 1.4 | 1.2 |
| M4 | 333.05 | 30 | 1.736 | 36 | 0.5346 | 36 | 1.2 | 1.2 |

*The values of β and γ were calculated from equation 11.

To obtain a final concentration c^{g-f} in a total volume of 50 mL, we calculate the needed mass of chitosan (f_{D}=0.866) with a loss on drying (L_{D}) of 0.1329 (water content) using this equation

$${m}_{c} = \frac{[ 161.1 \cdot {f}_{D} + 203.1 \cdot ( 1 - {f}_{D} ) ] \cdot {c}_{c}^{g - f} \cdot {V}_{T}}{1 - {L}_{D}} \cdot \frac{1}{{f}_{D}} \qquad ( 18 )$$

In a volumetric flask of 25 mL, we add the mass m_{c} of chitosan in about 20 mL of a H₂Odd-D₂O mix (11:2) and disperse the powder by stirring the solution. We add the volume V_{HCl} of HCl (1.037 M) and complete the volume to the mark using the H₂Odd-D₂O mix. We stir the solution with a magnetic bar overnight to obtain a 2×c_{c}^{g-f} and 2×c_{HCl}^{f} solution.

With a 25 mL-volumetric flask, we dissolved the mass m_{GP} of glycerol 2-phosphate into about 20 mL of the H₂Odd-D₂O mix. The solution was stirred until dissolution and complete the volume to the mark with H₂Odd-D₂O mix obtaining a 2×c_{GP}^{f} solution.

In a graduate cylinder of 50 mL, 20 mL of the prepared chitosan solution was added and the volume completed to 40 mL with the GP solution. Thus the two solutions are combined at a 1:1 ratio. A stir bar was added and the solution stirred for 10 minutes.

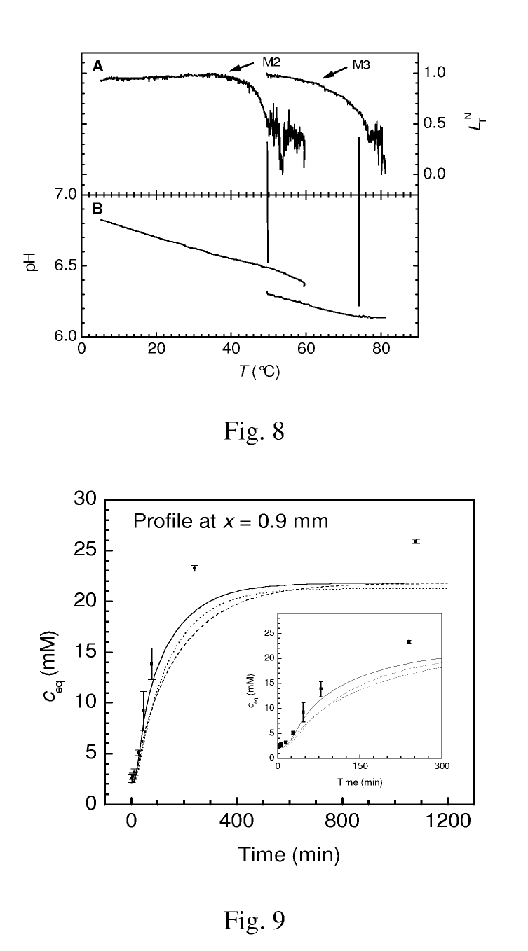

The phosphate chemical shift of prepared solutions (Table 5) was measured at 5, 15, and 37° C. and the value of χ found using equations 17 and 12. The value of χ is then used to calculate α, the degree of ionization of chitosan that was found to decrease with increasing temperature (FIG. 7) allowing chitosan precipitation and phase separation to occur (FIG. 8).

### Detailed Description of the Proof of Absence of Cross-Links Between the Polyelectrolyte and the Weak Electrolyte

Here we provide evidence that supports the notion that there is no ionic cross-link between the polyelectrolyte and the counter-ion, using gels kept in contact with a bath solution to allow the weak electrolyte (glycerol 2-phosphate in this example) to diffuse out of the gel. The counter-ion concentration was measured at different times in the bath solution and showed an increase in concentration with time. A simple diffusion model predicted the diffusion profile and allowed the calculation of the diffusion constant in the gel (see section “Diffusion from a gel to a washing solution”) showing an absence of binding with the chitosan gel component.

A 7.5 mL solution (see Table 6) containing chitosan (2.93% w/v Protosan UP CL 213) was placed on ice at 4° C. To this solution, we added 2.25 mL glucosamine (2.16% w/v) drop by drop every 15 seconds followed by 1 mL glycerol 2-phosphate (GP) (33.3% w/v) and 2 mL of hydroxyethyl cellulose. Approximately 5 g of this solution was poured into 5.3 cm diameter Petri dishes and placed in an incubator at 37° C. at 5% CO₂ under 100% relative humidity for 30 minutes. Another aliquot was taken for determination of initial phosphorus concentration in the gel.

TABLE 6
Composition of gel preparation where the solute mass mₛ is
dissolved in volume V_{b} of solvent. The volume Vₛ correspond
to the volume used for the preparation.

| | | Cat. | mₛ | | V_{b} | Vₛ |
|---|---|---|---|---|---|---|
| Solute | Company | No. | mg | Solvent | mL | mL |
| Protosan UP* | Pronova | CL 213 | 220.0 | ddH₂O | 7.5 | 7.5 |
| Glucosamine | Sigma | G-1514 | 53.9 | 0.1N NaOH | 2.5 | 2.25 |
| GP | Sigma | G-9891 | 800.0 | ddH₂O | 2** | 1 |
| Hydroxyethyl | Fluka | 54290 | 75.0 | DMEM | 3 | 2 |
| cellulose | | | | (pH = 7.4) | | |

*This chitosan is under a salt form. Therefore does not need HCl to be dissolved.
**Note that the final volume of this solution is 2.4 mL because the 800.0 mg of GP increase the volume by 0.4 mL.

The initial concentration of phosphorus in the gel is then (see Table 6)

$$\frac{800 mg \cdot \frac{1 mL}{2.4 mL}}{297 \frac{g}{mol} 12 \text{,} 75 mL} + 0 \text{,} 001 \frac{mol}{L} \cdot \frac{2 \text{,} 0 mL}{12 \text{,} 75 mL} = 0 \text{,} 0881 M \qquad ( 19 )$$

15.5 mL of DMEM (pH=7.4) was then layered over solidified gels in the Petri dishes containing gels and this time was defined as time zero, t₀. Aliquots (50 μL) of the DMEM bath medium were taken at predetermined times t_{P} (2.5, 6.5, 14.5, 28.0, 46.5, 79.0, 240.0 and 1080.0 minutes) for phosphorus content determination. The remaining DMEM solution was then removed and replaced by fresh DMEM for 240 minutes and again replaced by fresh DMEM for another 72 minutes.

Samples of gel were then taken for analysis of phosphorus content and additional gel samples also taken following three subsequent washes in DMEM for 60 minutes each. Phosphorus was quantified using the established method of Kjeldahl digestion (Liao, N., Total phosphorus in Kjeldahl digests, Milwaukee, Wis.: LACHAT Instruments, 25 p., QuickChem Method 10-115-01-1-C, 1993) followed by the analysis of the absorption at 880 nm of the PO₄³⁻ complex with ammonium molybdate and antimony potassic tartrate.

Concentration cₚ^{m} is the measured concentration from the absorption at 880 nm. Knowing that following the Kjeldahl digestion, a 21 mL solution is used to dilute phosphorus extracted from the V, aliquot volume, aliquots concentration measurements are given by

$${c}_{e} = \frac{21 \cdot 1000 \cdot {c}_{P}^{m}}{{V}_{e} \cdot 30 \text{,} 97} \qquad ( 20 )$$

Where 30.97 g/mol is the phosphorus molar mass.

To model the GP diffusion from the gel to the washing solution, we use a Cartesian representation where the x-axis originates at the bottom (x=0) of the Petri dish and is directed towards the top (FIG. 11).

The model presented in the section entitled “Diffusion from a gel to a washing solution” is used to calculate the concentration c(x,t) where c is the GP concentration at position x at time t. We also know the gel-solution interface position x=h_{g} and the solution-air interface position δ. The value h_{g} is determine from the Petri dish diameter dₚ and the gel volume. Approximating the gel volume from a density ρ_{g} taken to be 1 g/mL, the gel height h_{g} is given from its measured mass m_{g} using the following formula

$${h}_{g} = \frac{4 {m}_{g}}{{ρ}_{g} π {d}_{p}^{2}} \qquad ( 21 )$$

Knowing the washing solution volume V_{w} added on top of the gel, we find δ

$$δ = {h}_{g} + \frac{4 {V}_{w}}{π {d}_{p}^{2}} \qquad ( 22 )$$

It should be noted that during the equilibrium process, we observe a contraction of the gel (its mass m_{g} is lower after washing, meaning that water left the gel). To simulate this contraction, we describe h_{g} using a function of time

$${h}_{g} ( t ) = {h}_{g}^{c} + \frac{{h}_{g}^{i} - {h}_{g}^{c}}{1 + {e}^{\frac{( t - {t}_{0.5} )}{τ}}} \qquad ( 23 )$$

where h_{g}ⁱ represents the initial gel thickness and h_{g}^{c} represents its contracted value. The time t_{0.5} represents the time for the gel to reach its half-contraction and τ is a time relative to its contraction speed.

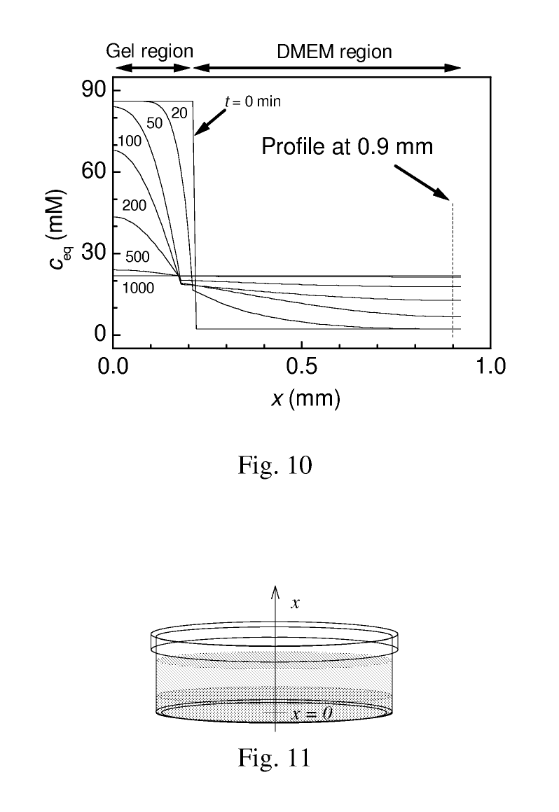

FIG. 9 shows the concentration of GP in the DMEM bath solution over time along with model predictions (lines) that assume free diffusion. The close coincidence of the model prediction to measurements clearly indicates lack of binding of GP to the components of the gel. FIG. 10 shows GP concentration profiles predicted by the model within the gel and in the solution in the Petri dish. Moreover, Table 7 shows different phosphorus concentration values obtained from different samples where the initial concentration is 86 mM and reaches the DMEM concentration of about 2 mM.

TABLE 7
Determination of the total phosphorus concentration (from the measured
concentration cₚ^{m}) in the aliquot of volume Vₑ and the corresponding
concentration cₑ in the sample.

| | c_{P}^{m} | Vₑ | cₑ |
|---|---|---|---|
| Sample | mg/L | μL | mM |
| Initial gel | 26.41 | 207.9* | 86.1 |
| DMEM (pH = 7.4) | 0.16 | 50 | 2.16 |
| Gel after 3 washes | 0.10 | 22.8* | 2.97 |
| Gel after 6 washes | 0.05 | 21.7* | 1.56 |

The Degree of Ionization of Polyelectrolytes in Solution Varies with Temperature in a Manner Predicted by the Temperature-Dependence of their Dissociation Constants

Since β and γ vary between 0 and 1 and β is positive, it follows from equation 12 that γ respects the following conditions

β−1<γ<2β (24)

We now replace χ by a function of Kₐ^{Glc}, Kₐ₂^{GP} and γ using equations 7 and 8, and the parameters defined in equation 11. The dissociation equations 1 and 3 can now be rewritten as

$$\begin{matrix}{K}_{ap}^{Glc} = \frac{{c}_{{H}^{+}} ( 1 - α )}{α} & ( 25 ) \\ {K}_{a 2}^{P} = \frac{{c}_{{H}^{+}} χ}{1 - χ} & ( 26 )\end{matrix}$$

We define

$$R = \frac{{K}_{a}^{Glc}}{{K}_{a}^{GP}} \qquad ( 27 )$$

Dividing equation 25 by equations 26 and 27, we eliminate c_{H}_{⁺} and obtain

$$\frac{( 1 - χ ) ( 1 - α )}{χα} = R \\ ⇔ \\ \frac{1}{χ} = \frac{α R}{( 1 - α )} + 1 \\ ⇔ \\ χ + 1 = \frac{2 ( 1 - α ) + α R}{( 1 - α ) + α R} \qquad ( 28 )$$

The term χ+1 can now be substituted into 12 using equation 28. We eliminate c_{GPO}_{₄}_{^{2−}} and obtain a quadratic equation for α

$$α + γ = β \frac{2 ( 1 - α ) + α R}{( 1 - α ) + α R} \\ ⇔ \\ α - {α}^{2} + {α}^{2} R + γ - αγ + α γ R = 2 β - 2 α β + αβ R \\ ⇔ \\ {α}^{2} ( R - 1 ) + α ( 2 β - γ + 1 + R ( γ - β ) ) - ( 2 β - γ + 1 - 1 ) = 0 \qquad ( 29 )$$

In order to simplify the following calculations, we define two parameters

A=2β−γ+1 (30)

B=γ−β (31)

Such that equation 29 becomes

α²(R−1)+α(A+B·R)−(A−1)=0 (32)

providing α is

$$α = \frac{- ( A + B \cdot R ) \pm \sqrt{{( A + B \cdot R )}^{2} + 4 ( R - 1 ) ( A - 1 )}}{2 ( R - 1 )} \qquad ( 33 )$$

Since A and B are constants (they are only functions of β and γ), the conditions for γ (equation 24) become

1<A<β+2 (34)

−1<B<β (35)

From A+B=β+1, we also have

A+B>1 (36)

We can show from equation 36 that appropriate root of equation 33 is that with the positive sign before the square root. The square root term of equation 33 can be rewritten

$$\begin{matrix}{( A + B \cdot R )}^{2} + 4 ( R - 1 ) ( A - 1 ) = {A}^{2} + 2 A \cdot B \cdot R + {B}^{2} {R}^{2} + \\ 4 A \cdot R - 4 A - 4 R + 4 \\ = {( A - 2 )}^{2} + {B}^{2} {R}^{2} + \\ 2 R ( A \cdot B + 2 A - 2 ) \\ = {( A - 2 + B \cdot R )}^{2} - \\ 2 ( A - 2 ) B \cdot R + \\ 2 R ( A \cdot B + 2 A - 2 ) \\ = {( A - 2 + B \cdot R )}^{2} + \\ 4 R ( A + B - 1 ) > 0\end{matrix} \qquad ( 37 )$$

From equation 32 and the condition A>1 (equation 34) and α>0, we also have

α(R−1)+(A+B·R)>0 (38)

From equation 32 we see that if R<1, we have that (A+B·R) is positive and greater than α(1−R). Therefore, examining equation 33, we see that the positive root must be taken to respect condition 38. Moreover, if R>1, we know that equation 33 is greater than A+B·R (since A>1) and we see that the positive root is taken to obtain α>0. Therefore, α is given by the positive root of equation 33.

We now need to find the partial derivative of equation 33 with respect to the temperature T. We first define

$$\begin{matrix}x = A + BR & ( 39 ) \\ \begin{matrix}y = {( A + B \cdot R )}^{2} + 4 ( R - 1 ) ( A - 1 ) \\ = {( A - 2 )}^{2} + {B}^{2} {R}^{2} + 2 ( AB + 2 A - 2 ) R\end{matrix} & ( 40 ) \\ z = ( R - 1 ) & ( 41 )\end{matrix}$$

We then have

$$α = \frac{- x + \sqrt{y}}{2 z} \qquad ( 42 )$$

The partial derivative of the previous equation with respect to the temperature T is then

$$\begin{matrix}\frac{\partial α}{\partial T} = \frac{1}{2 {z}^{2}} ( - z \frac{\partial x}{\partial T} + \frac{z}{2 \sqrt{y}} \frac{\partial y}{\partial T} + ( x - \sqrt{y} ) \frac{\partial z}{\partial T} ) \\ = \frac{1}{2 {z}^{2} \sqrt{y}} ( - z \sqrt{y} \frac{\partial x}{\partial T} + \frac{z}{2} \frac{\partial y}{\partial T} + ( x \sqrt{y} - y ) \frac{\partial z}{\partial T} ) \\ = \frac{1}{2 {z}^{2} \sqrt{y}} ( \sqrt{y} ( x \frac{\partial z}{\partial T} - z \frac{\partial x}{\partial T} ) + \frac{z}{2} \frac{\partial y}{\partial T} - y \frac{\partial z}{\partial T} )\end{matrix} \qquad ( 43 )$$

The derivatives of x, y and z give

$$\begin{matrix}\frac{\partial x}{\partial T} = B \cdot \frac{\partial R}{\partial T} & ( 44 ) \\ \frac{\partial y}{\partial T} = 2 ( {B}^{2} R + AB + 2 A - 2 ) \frac{\partial R}{\partial T} & ( 45 ) \\ \frac{\partial z}{\partial T} = \frac{\partial R}{\partial T} & ( 46 )\end{matrix}$$

Using equations 39, 41, 44 et 46, the term with the derivatives of x and z in equation 43 are

$$\begin{matrix}x \frac{\partial z}{\partial T} - z \frac{\partial x}{\partial T} = ( A + B \cdot R ) \frac{\partial R}{\partial T} - ( R - 1 ) B \frac{\partial R}{\partial T} \\ = ( A + B ) \frac{\partial R}{\partial T}\end{matrix} \qquad ( 47 )$$

Using equations 40, 41, 45 et 46, the term with the derivatives of y et z in equation 43 are

$$\begin{matrix}\frac{z}{2} \frac{\partial y}{\partial T} - y \frac{\partial z}{\partial T} = ( R - 1 ) ( {B}^{2} R + AB + 2 A - 2 ) \frac{\partial R}{\partial T} - \\ ( {( A - 2 )}^{2} + {B}^{2} {R}^{2} + 2 ( AB + 2 A - 2 ) R ) \frac{\partial R}{\partial T} \\ = ( \begin{matrix}{B}^{2} {R}^{2} + ABR + 2 AR - \\ 2 R - {B}^{2} R - AB - 2 A + 2 - \\ {A}^{2} + 4 A - 4 - {B}^{2} {R}^{2} - \\ 2 ABR - 4 AR + 4 R\end{matrix} ) \frac{\partial R}{\partial T} \\ = - ( R ( {B}^{2} + AB + 2 A - 2 ) + \\ {A}^{2} + AB - 2 A + 2 ) \frac{\partial R}{\partial T}\end{matrix} \qquad ( 48 )$$

We define the coefficient of

$$- \frac{\partial R}{\partial T}$$

in the right-hand term of this previous equation equal to f and rewrite this term as

$$\begin{matrix}f = ( {A}^{2} + AB - 2 A + 2 ) + R ( {B}^{2} + AB + 2 A - 2 ) \\ = ( A + B - 1 ) ( A - 1 ) + ( B + 1 ) + \\ R ( A + B - 1 ) ( B + 1 ) + R ( A - 1 ) \\ = ( A - 1 ) [ ( A + B ) + ( R - 1 ) ] + ( B + 1 ) [ R ( A + B ) - ( R - 1 ) ]\end{matrix} \qquad ( 49 )$$

From equation 43 and relations 47, 48 and 49 the derivative of α with respect to the temperature T is given by

$$\frac{\partial α}{\partial T} = \frac{1}{2 {z}^{2} \sqrt{y}} ( ( A + B ) \sqrt{y} - f ) \frac{\partial R}{\partial T} \qquad ( 50 )$$

Also, we have

$$\begin{matrix}- log R = - log \frac{{K}_{ap}^{Glc}}{{K}_{a 2}^{GP}} = {pK}_{ap}^{Glc} - {pK}_{a 2}^{GP} & ( 51 ) \\ \frac{\partial ( - log R )}{\partial T} = - \frac{1}{ln 10 R} \frac{\partial R}{\partial T} & ( 52 )\end{matrix}$$

Therefore, we can rewrite equation 50 to obtain

$$\frac{\partial α}{\partial T} = \frac{ln 10 {K}_{a}^{Glc}}{2 {z}^{2} \sqrt{y} {K}_{a 2}^{GP}} ( f - ( A + B ) \sqrt{y} ) ( \frac{\partial {pK}_{a}^{Glc}}{\partial T} - \frac{\partial {pK}_{a 2}^{GP}}{\partial T} ) \qquad ( 53 )$$

In order to obtain a reduction of the ionization degree of the polyelectrolyte we require conditions such that

$$\frac{\partial α}{\partial T} < 0.$$

We now need to know if f−(A+B)\sqrt{y}>0, that is equivalent to showing f²−(A+B)²y>0 since (A+B)\sqrt{y}>0 (see equation 36). We have for f², from equation 49,

$$\begin{matrix}\begin{matrix}{f}^{2} = {{( A - 1 )}^{2} [ ( A + B ) + ( R - 1 ) ]}^{2} + \\ 2 ( A - 1 ) ( B + 1 ) [ ( A + B ) + ( R - 1 ) ] [ \begin{matrix}R ( A + B ) - \\ ( R - 1 )\end{matrix} ] + \\ {{( B + 1 )}^{2} [ R ( A + B ) - ( R - 1 ) ]}^{2}\end{matrix} \\ = {( A - 1 )}^{2} [ \begin{matrix}{( A + B )}^{2} + 2 ( A + B ) \\ ( R - 1 ) + {( R - 1 )}^{2}\end{matrix} ] + \\ 2 ( A - 1 ) ( B + 1 ) [ \begin{matrix}\begin{matrix}{R ( A + B )}^{2} - \\ ( R - 1 ) ( A + B ) +\end{matrix} \\ R ( R - 1 ) ( A + B ) + {( R - 1 )}^{2}\end{matrix} ] + \\ {( B + 1 )}^{2} [ \begin{matrix}{{R}^{2} ( A + B )}^{2} - 2 R ( A + B ) \\ ( R - 1 ) + {( R - 1 )}^{2}\end{matrix} ] & ( 54 ) \\ \begin{matrix} = {( A - 1 )}^{2} [ \begin{matrix}{( A + B )}^{2} + 2 ( A + B ) \\ ( R - 1 ) + {( R - 1 )}^{2}\end{matrix} ] + \\ 2 ( A - 1 ) ( B + 1 ) [ \begin{matrix}{R ( A + B )}^{2} + \\ ( A + B - 1 ) {( R - 1 )}^{2}\end{matrix} ] + \\ {( B + 1 )}^{2} [ \begin{matrix}{{R}^{2} ( A + B )}^{2} - \\ 2 R ( A + B ) ( R - 1 ) + {( R - 1 )}^{2}\end{matrix} ]\end{matrix} & \\ \begin{matrix} = {( A + B )}^{2} [ \begin{matrix}{( A - 1 )}^{2} + 2 R ( A - 1 ) \\ ( B + 1 ) + {{R}^{2} ( B + 1 )}^{2}\end{matrix} ] + \\ {( R - 1 )}^{2} [ \begin{matrix}{( A - 1 )}^{2} + 2 ( A - 1 ) ( B + 1 ) \\ ( A + B - 1 ) + {( B + 1 )}^{2}\end{matrix} ] + \\ 2 ( A + B ) ( R - 1 ) [ {( A - 1 )}^{2} - {R ( B + 1 )}^{2} ]\end{matrix} \\ \begin{matrix} = {{( A + B )}^{2} [ ( A - 1 ) + R ( B + 1 ) ]}^{2} + \\ {( R - 1 )}^{2} [ \begin{matrix}{( ( A - 1 ) - ( B + 1 ) )}^{2} + \\ 2 ( A - 1 ) ( B + 1 ) ( A + B )\end{matrix} ] + \\ 2 ( A + B ) ( R - 1 ) [ {( A - 1 )}^{2} - {R ( B + 1 )}^{2} ]\end{matrix} & \\ Therefore , & \\ \begin{matrix}{f}^{2} - {( A + B )}^{2} y = {{( A + B )}^{2} [ ( A - 1 ) + R ( B + 1 ) ]}^{2} + \\ {( R - 1 )}^{2} [ \begin{matrix}{( ( A - 1 ) - ( B + 1 ) )}^{2} + \\ 2 ( A - 1 ) ( B + 1 ) ( A + B )\end{matrix} ] + \\ 2 ( A + B ) ( R - 1 ) {( A - 1 )}^{2} - \\ 2 R ( A + B ) ( R - 1 ) {( B + 1 )}^{2} - \\ {( A + B )}^{2} [ {( A + B \cdot R )}^{2} + 4 ( R - 1 ) ( A - 1 ) ] \\ = {( A + B )}^{2} [ \begin{matrix}{( ( A + RB ) + ( R - 1 ) )}^{2} - \\ {( A + RB )}^{2}\end{matrix} ] + \\ {( R - 1 )}^{2} [ \begin{matrix}{( ( A - 1 ) - ( B + 1 ) )}^{2} + \\ 2 ( A - 1 ) ( B + 1 ) ( A + B )\end{matrix} ] + \\ ( R - 1 ) ( A - 1 ) ( A + B ) \\ [ 2 ( A - 1 ) - 4 ( A + B ) ] - \\ 2 R ( A + B ) ( R - 1 ) {( B + 1 )}^{2}\end{matrix} & \\ \begin{matrix} = {( A + B )}^{2} [ 2 ( A + RB ) ( R - 1 ) + {( R - 1 )}^{2} ] + \\ {( R - 1 )}^{2} [ \begin{matrix}{( ( A - 1 ) - ( B + 1 ) )}^{2} + \\ 2 ( A - 1 ) ( B + 1 ) ( A + B )\end{matrix} ] - \\ 2 ( R - 1 ) ( A - 1 ) ( A + B ) ( A + 2 B + 1 ) - \\ 2 R ( A + B ) ( R - 1 ) {( B + 1 )}^{2} \\ = 2 ( R - 1 ) ( A + B ) [ \begin{matrix}( A + RB ) ( A + B ) - \\ ( A - 1 ) ( A + 2 B + 1 ) - \\ {R ( B + 1 )}^{2}\end{matrix} ] + \\ {( R - 1 )}^{2} [ \begin{matrix}{( ( A - 1 ) - ( B + 1 ) )}^{2} + \\ 2 ( A - 1 ) ( B + 1 ) ( A + B ) + \\ {( A + B )}^{2}\end{matrix} ]\end{matrix} & \\ \begin{matrix} = 2 ( R - 1 ) ( A + B ) [ \begin{matrix}( \begin{matrix}{A}^{2} + RAB + \\ AB + {RB}^{2}\end{matrix} ) - \\ ( \begin{matrix}{A}^{2} + 2 AB - \\ 2 B - 1\end{matrix} ) - \\ R ( {B}^{2} + 2 B + 1 )\end{matrix} ] + \\ {( R - 1 )}^{2} [ \begin{matrix}{( ( A - 1 ) - ( B + 1 ) )}^{2} + \\ {( A + B )}^{2} + 2 ( A - 1 ) \\ ( B + 1 ) ( A + B )\end{matrix} ] \\ = 2 ( R - 1 ) ( A + B ) [ \begin{matrix}RAB - \\ ( AB - 2 B - 1 ) - \\ R ( 2 B + 1 )\end{matrix} ] + \\ {( R - 1 )}^{2} [ \begin{matrix}{( ( A - 1 ) - ( B + 1 ) )}^{2} + \\ {( A + B )}^{2} + 2 ( A - 1 ) \\ ( B + 1 ) ( A + B )\end{matrix} ]\end{matrix} & \\ \begin{matrix} = 2 {( R - 1 )}^{2} ( A + B ) ( AB - 2 B - 1 ) + \\ {( R - 1 )}^{2} [ \begin{matrix}{( A - 1 )}^{2} - 2 ( A - 1 ) ( B + 1 ) + \\ {( B + 1 )}^{2} + {( A + B )}^{2} + 2 ( A - 1 ) \\ ( B + 1 ) ( A + B )\end{matrix} ] \\ = 2 {( R - 1 )}^{2} ( A + B ) [ \begin{matrix}( AB + A - B - 1 ) - \\ ( A + B )\end{matrix} ] + \\ {( R - 1 )}^{2} [ \begin{matrix}{( A - 1 )}^{2} - 2 ( A - 1 ) ( B + 1 ) + \\ {( B + 1 )}^{2} + {( A + B )}^{2} + 2 ( A - 1 ) \\ ( B + 1 ) ( A + B )\end{matrix} ] \\ = 2 {( R - 1 )}^{2} ( A - 1 ) ( B + 1 ) ( A + B ) + \\ {( R - 1 )}^{2} [ \begin{matrix}{( A - 1 )}^{2} - 2 ( A - 1 ) ( B + 1 ) + \\ {( B + 1 )}^{2} - 2 {( A + B )}^{2} + \\ {( A + B )}^{2} + 2 ( A - 1 ) ( B + 1 ) \\ ( A + B )\end{matrix} ]\end{matrix} & \\ \begin{matrix} = {( R - 1 )}^{2} [ \begin{matrix}{( A - 1 )}^{2} - 2 ( A - 1 ) ( B + 1 ) + \\ {( B + 1 )}^{2} - {( A + B )}^{2} + \\ 4 ( A - 1 ) ( B + 1 ) ( A + B )\end{matrix} ] \\ = {( R - 1 )}^{2} [ \begin{matrix}[ ( A - 1 ) - {( B + 1 )}^{2} ] - \\ {( A + B )}^{2} + 4 ( A - 1 ) \\ ( B + 1 ) ( A + B )\end{matrix} ] \\ = {( R - 1 )}^{2} [ \begin{matrix}{[ A - B - 2 ]}^{2} - {( A + B )}^{2} + \\ 4 ( A - 1 ) ( B + 1 ) ( A + B )\end{matrix} ] \\ = {( R - 1 )}^{2} [ \begin{matrix}[ ( A - B - 2 ) + ( A + B ) ] \\ [ ( A - B - 2 ) - ( A + B ) ] + \\ 4 ( A - 1 ) ( B + 1 ) ( A + B )\end{matrix} ] \\ = {( R - 1 )}^{2} [ \begin{matrix}( 2 A - 2 ) ( - 2 B - 2 ) + \\ 4 ( A - 1 ) ( B + 1 ) ( A + B )\end{matrix} ] \\ = 4 {( R - 1 )}^{2} ( A - 1 ) ( B + 1 ) ( A + B - 1 )\end{matrix} & \end{matrix}$$

And therefore,

f>(A+B)\sqrt{y}>0 (55)

And the condition producing

$$\frac{\partial α}{\partial T} < 0 is \frac{\partial {pK}_{ap}^{Glc}}{\partial T} < \frac{\partial {pK}_{a 2}^{GP}}{\partial T} \qquad ( 56 )$$

because

$$\frac{ln 10 {K}_{a}^{Glc}}{2 {z}^{2} \sqrt{y } {K}_{a 2}^{GP}} ( f - ( A + B ) \sqrt{y} ) > 0.$$

Derivation of dpKₐₚ/dpH≅1

Assuming an ideal solution the equilibrium constant of the cationic polyelectrolyte dissociation AH⁺ [symbol] H⁺+A is

$${K}_{a} = \frac{{c}_{{H}^{+}} {c}_{A}}{{c}_{{AH}^{+}}} \qquad ( 57 )$$

and water dissociation to protons and hydroxyl ions related by

K_{w}=c_{H}_{⁺}c_{OH}_{^{−}} (58)

Conservation of the total number of ionizable sites requires

c_{HA}^{total}=c_{HA}_{⁺}+c_{A} (59)

Solution electroneutrality in the presence of a strong base and/or acid like NaOH or HCl is

$${\sum }_{i} {z}_{i} {c}_{i} = {c}_{{Na}^{+}} + {c}_{{HA}^{+}} + {c}_{{H}^{+}} - {c}_{{Cl}^{-}} - {c}_{{OH}^{-}} = 0 \qquad ( 60 )$$

where zᵢ is the valence of species i and cᵢ is its concentration.

The degree of ionization

$$α = \frac{{c}_{{HA}^{+}}}{{c}_{HA}^{total}} \qquad ( 61 )$$

combined with equation 59 provides

$$\frac{{c}_{A}}{{c}_{HA}^{total}} = 1 - α \qquad ( 62 )$$

Approximating γ_{H}_{⁺}=1 in the definition of pH=−log₁₀ ā_{H}=−log₁₀ γ₊ c̅_{H} with pKₐ=−log₁₀ Kₐ, equation 57 combined with equations 61 and 62 provides

$${pK}_{a} = pH - {log}_{10} \frac{1 - α}{α} \qquad ( 63 )$$

Differentiating equation 63 we have

$$\frac{d {pK}_{a}}{d pH} = 1 + \frac{1}{ln 10} \cdot \frac{1}{α ( 1 - α )} \cdot \frac{d α}{d pH} \qquad ( 64 )$$

while equation 61 and pH=−log₁₀ c̅_{H} provide

$$d α = \frac{d {c}_{{HA}^{+}}}{{c}_{HA}^{total}} and d pH = - \frac{1}{{c}_{{H}^{+}}} \cdot \frac{d {c}_{{H}^{+}}}{ln 10} \qquad ( 65 )$$

Taking into account that the strong base and/or acid are entirely dissociated such that

$$\frac{d {c}_{{Na}^{+}}}{d {c}_{{H}^{+}}} = \frac{d {c}_{{Cl}^{-}}}{d {c}_{{H}^{+}}} = 0 \qquad ( 66 )$$

we find by differentiating electroneutrality equation 60 with respect to C_{H}_{⁺} that

$$\frac{d {c}_{{OH}^{-}} - d {c}_{{HA}^{+}}}{d {c}_{{H}^{+ }}} = 1 \qquad ( 67 )$$

Differentiating water dissociation equation 58 similarly provides

$$\frac{d {c}_{{OH}^{-}}}{d {c}_{{H}^{+ }}} = - \frac{{c}_{{OH}^{-}}}{{c}_{{H}^{+}}} \qquad ( 68 )$$

Inserting equations 65 to 68 into equation 64 results in

$$\begin{matrix}\frac{d {pK}_{a }}{d pH} = 1 + \frac{1}{α ( 1 - α )} \frac{{c}_{{H}^{+}}}{{c}_{HA}^{total}} ( 1 + \frac{{c}_{{OH}^{-}}}{{{c}_{H}}^{+}} ) \\ = 1 + \frac{1}{α ( 1 - α )} \frac{{c}_{{H}^{+}} + {c}_{{OH}^{-}}}{{c}_{HA}^{total}}\end{matrix} \qquad ( 69 )$$

where use of equations 59, 61 and 62, reveals

$$\begin{matrix}\frac{d {pK}_{a}}{d pH} = 1 + \frac{{c}_{HA}^{total} {c}_{{H}^{+}}}{{c}_{{HA}^{+}} {c}_{A}} ( 1 + \frac{{c}_{{OH}^{-}}}{{c}_{{H}^{+}}} ) \\ = 1 + ( {c}_{{H}^{+}} + {c}_{{OH}^{-}} ) ( \frac{1}{{c}_{{HA}^{+}}} + \frac{1}{{c}_{A}} )\end{matrix} \qquad ( 70 )$$

Finally we find that

$$\begin{matrix}\frac{d {pK}_{a}}{d pH} ≅ 1 \\ if & ( 71 ) \\ ( {c}_{{H}^{+}} + {c}_{{OH}^{-}} ) ( \frac{1}{{c}_{{HA}^{+}}} + \frac{1}{{c}_{A}} ) << 1 & ( 72 )\end{matrix}$$

Poisson-Boltzmann Cylindrical Cell Model Predicts pKₐₚ Variation with the Degree of Ionization for a Polyelectrolyte

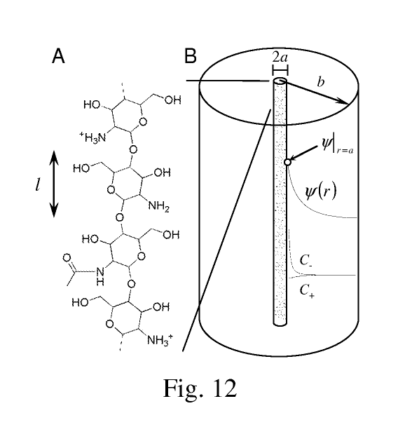

Chitosan is composed of two distinct monomers: a fraction f_{D} of ionizable glucosamine and a fraction 1−f_{D} of nonionizable N-acetyl-glucosamine (FIG. 12A). The chitosan is represented as an infinite impenetrable cylinder of radius a where discrete charge sites are smeared out to form a uniform surface charge density σ(FIG. 12B),

$$σ = \frac{e α {f}_{D}}{2 π al} \qquad ( 73 )$$

where e is the elementary charge, α is the degree of ionization of the polycation (α=0 is neutral and α=1 is fully ionized) and l is the structural length of the monomer that is set to l=0.52 nm following structural data (Mazeau et al., Macromolecules 27: 7606-7612, 1994; Okuyama et al., Macromolecules 30: 5849-5855, 1997). The radius of the inner cylinder representing chitosan is taken as a=0.8 nm. Each polymer chain is located at the center of a cylindrical cell whose radius b (FIG. 12A) is determined from the monomer concentration cₚ (including both glucosamine and N-acetyl-glucosamine) and monomer length l, according to

$$b = {( \frac{1}{π l {c}_{p} {N}_{A }} )}^{1 / 2} \qquad ( 74 )$$

where N_{A} is Avogadro's number.

The polycation is surrounded by mobile ions in the region a<r<b. Using the mean field approximation (Marcus, R. A., J Chem Physics 23: 1057-1068, 1955), these ions are assumed to follow a Boltzmann distribution at equilibrium, resulting in a concentration profile cᵢ(r) about the poly ion that is a function of radial position r and electrostatic potential ψ(r),

cᵢ(r)=cᵢ⁰e^{-z}_{ᵢ}^{eψ(r)/kT} (75)

where zᵢ is the valence of the mobile ionic species i, T is the temperature, and k is Boltzmann's constant. The position where the electrostatic potential is zero, and therefore where cᵢ⁰ would be the concentration of ionic species i, always exist in a solution where the polymer is infinitely dilute or when the solution is in equilibrium across a semi-permeable membrane (permeable to salt but not to the polyelectrolyte). When these conditions do not apply, as in the case of a closed polyelectrolyte solution at finite concentration in the present study where ψ>0 can occur throughout the solution, then the value of cᵢ⁰ can be found from the known mean concentration of positive or negative electrolyte ions, c̅_{±}, in the volume of the cylindrical cell that are given by

$${\overline{c}}_{\pm } = {c}_{\pm }^{0} \frac{{\int }_{a}^{b} 2 r {e}^{∓ \frac{e ψ ( r )}{kT}} d r}{{b}^{2}} = \frac{{c}_{\pm }^{0}}{{γ}_{\pm }} \qquad ( 76 )$$

where a mono-monovalent electrolyte, zᵢ=1+ or 1−, is considered and

$${γ}_{\pm } = \frac{{b}^{2}}{{\int }_{a}^{b} 2 r {e}^{\pm \frac{e ψ ( r )}{kT}} d r}$$

are the mobile ion activity coefficients in the cylindrical cell, particular to this mean-field theory.

A theoretical relationship describing pH dependence on pK₀, α and ψ|ᵣ₌ₐ where ψ|ᵣ₌ₐ is the electrostatic potential at the surface of the polyelectrolyte from the Poisson-Boltzmann cylindrical cell model (Marcus, R. A., J Chem Physics 23: 1057-1068, 1955) is given by

$$\begin{matrix}pH = - {log}_{10} {γ}_{+} {c}_{H} = {pK}_{0} ( T ) + {log}_{10} \frac{1 - α}{α} - \frac{{e ψ \vert}_{r = a}}{ln 10 kT} \\ where & ( 77 ) \\ {pK}_{0} ( T ) = \frac{{μ}_{H}^{0} + {μ}_{A}^{0} - {μ}_{{AH}^{+}}^{0}}{ln 10 kT} & ( 78 )\end{matrix}$$

and μ_{H}⁰ is the standard proton chemical potential in the solution phase, and μ_{AH}_{⁺}⁰ and μ_{A}⁰ are the standard chemical potentials of a protonated and a neutral site on the polycation, respectively. A useful expression to compare with experiments is the apparent pKₐ, or pKₐₚ

$${pK}_{ap} ( T ) = pH ( T ) - {log}_{10} \frac{1 - α}{α} = {pK}_{0} ( T ) - \frac{e ψ {\vert}_{r = a}}{ln 10 kT} \qquad ( 79 )$$

that includes two contributions, the first representing the intrinsic monomeric dissociation constant pK₀(T), and the second containing the polyelectrolyte surface potential ψ|ᵣ₌ₐ that can be found by solving the Poisson-Boltzmann equation. Note that for simple acid/base electrolytes ψ|ᵣ₌ₐ=0 in the current model and therefore the apparent pKₐ (pKₐₚ) and pKₐ become identical pKₐₚ(T)=pK₀(T)=pKₐ(n.

The use of equation 77 to determine pH requires knowledge of ψ|ᵣ₌ₐ, α, and pK₀ for a given temperature, T. The electrostatic potential, ψ(a<r<b), can be found from the solution to the Poisson-Boltzmann equation (Buschmann and Grodzinsky, J Biomech Eng 117: 179-192, 1995; Carnie and Torrie, Adv Chem Phys 56: 141-253, 1984; Fixman, J Chem Phys 70: 4995-5005, 1979) in cylindrical coordinates,

$$\frac{{d}^{2} ψ ( r )}{d {r}^{2}} + \frac{1}{r} \frac{d ψ ( r )}{d r} = - \frac{ρ ( r )}{ɛ} = - \frac{{\sum }_{i} {z}_{i} {e}_{i}^{0} {e}^{- {z}_{i} e ψ ( r ) / kT}}{ɛ} \qquad ( 80 )$$

subject to boundary conditions from Gauss' law

$${{\frac{d ψ ( r )}{d r } \vert}_{r = a } = - \frac{σ}{ɛ} = - \frac{e α {f}_{D}}{2 π al ɛ} and \frac{d ψ ( r )}{d r} \vert}_{r = b} = 0 \qquad ( 81 )$$

where ε is the permittivity of water and ρ(r) is the spatially varying charge density. In a region where the electrostatic potential and derivatives are zero (i.e. a real or virtual ground) we have from equation 80

$${\sum }_{i = mobile ions} {z}_{i} {ec}_{i}^{0} = 0 \qquad ( 82 )$$

In the context of this study, the mobile ions considered are the weak electrolyte Cl⁻ (from the solvent HCl and NaCl salt added), the co-ion Na⁺ (from the dissociation of NaOH and NaCl) and protons (H⁺). Hydroxyl ion (OH⁻) concentration is negligible since only acidic solutions are considered. Then equation 82 can be used to define a concentration of total cationic or total anionic species, c⁰, at the real or virtual ground as

c_{Na}⁰+cₕ⁰=c_{Cl}⁰=c⁰ (83)

Since both cations follow the same Boltzmann distribution, the summation on the right side of equation 80 can be written in terms of c_{Cl}⁰ alone, using equation 83, to obtain

$$\frac{{d}^{2} ψ ( r )}{d {r}^{2}} + \frac{1}{r} \frac{d ψ ( r )}{d r} = \frac{2 {ec}_{Cl}^{0}}{ɛ} sinh ( \frac{e ψ ( r )}{kT} ) \qquad ( 84 )$$

The experimentally known average Cl⁻ concentration c̅_{Cl} (the sum of HCl and NaCl concentrations) is then directly related to c_{Cl}⁰ via equation 76. Thus, for polycations containing monovalent salt at acidic pH, the electrostatic potential for a known degree of ionization α is found by numerically solving equation 84 such that the boundary conditions of equation 81 are satisfied, using an initial guess for c_{Cl}⁰ that is iterated until the right-hand side of equation 76 converges to the known concentration, c̅_{Cl}. In this way the Poisson-Boltzmann equation may be solved for a closed solution at finite polyelectrolyte concentration that is not in equilibrium with an external bath.

The degree of ionization, α is required to calculate pH from equation 77. To determine α, we use the condition of macroscopic electroneutrality, again assuming negligible amounts of hydroxyl ions,

c̅_{Cl}− c̅_{Na}− c̅_{H}−c_{g}⁺=0 (85)

where c_{g}⁺_{g} is the concentration of ionized glucosamine monomers,

c_{g}⁺=αf_{D}cₚ (86)

Substituting equations 77 and 86 into equation 85 we find

$$α = \frac{{\overline{c}}_{C l} - {\overline{c}}_{Na} - \frac{{10}^{- p H}}{{γ}_{+}}}{{f}_{D} {c}_{p}} \qquad ( 87 )$$

The value of α and the corresponding ψ|ᵣ₌ₐ are determined for each particular experimental pH. In most cases, the proton concentration is negligible and α is simply determined from the known ion and monomer concentrations (taking into account any dilution from the cumulative titrant addition). For cases where proton concentration must be considered (i.e. the term

$$\frac{{10}^{- p H}}{{γ}_{+}}$$

in equation 87 is significant), the degree of ionization α can be estimated by using the pH experimental value and assuming an activity coefficient equal to one. For low ionic strength and low pH values, the approximation γ₊=1 becomes inaccurate, in which case, after having solved the Poisson-Boltzmann equation (equation 84) as described in the previous section, the potential profile ψ(r) is used to calculate γ₊ using equation 76 and this γ₊ is subsequently inserted into equation 77 to obtain a new α and the process is iterated until a converges to a unique value.
Diffusion from a Gel to a Washing Solution

We define a concentration function c(x,t) for the counter-ion where x represents the position in the axial direction of the Petri dish and t represents the time (see FIG. 11). This function is a solution of the diffusion equation

$$\frac{\partial c ( x , t )}{\partial t} = D ( x , t ) \frac{{\partial }^{2} c ( x , t )}{{\partial }^{2} x} \qquad ( 88 )$$

Lack of diffusive flux at the impermeable boundaries requires

$$\frac{\partial c ( 0 , t )}{\partial x} = \frac{\partial c ( δ , t )}{\partial x} = 0 \qquad ( 89 )$$

The diffusion coefficient D(x,t) in the gel is D_{g}, while in the solution an larger coefficient Dₛ is chosen to account for stirring. Therefore

$$\begin{matrix}D ( x , t ) = {D}_{g} & pour & x < {h}_{g} ( t ) \\ = {D}_{s} & pour & x > {h}_{g} ( t )\end{matrix} \qquad ( 90 )$$

Initial conditions are

$$\begin{matrix}c ( x , 0 ) = {c}_{g} & pour & x < {h}_{g} ( 0 ) \\ = {c}_{s} & pour & x > {h}_{g} ( 0 )\end{matrix} \qquad ( 91 )$$

where c_{g} and cₛ are phosphate concentration values in the gel and the solution, respectively.

The diffusion equation (equation 88) was solved respecting the specified conditions (equations 89 to 91) using the pdepe function from MatLab software.

The present invention will be more readily understood by referring to the following examples, which are given to illustrate the invention rather than to limit its scope.

### Example 1

### Preparation of a Thermo Sensitive Chitosan Phosphate Gel

Now using the previous equations, it is now possible to arrive more rapidly at a gel composition comprising a solution of 2% w/v of chitosan with a degree of deacetylation of 78.5% dissolved in HCl 0.092 M. The solution is stirred vigorously for about 2 hours in order to dissolve the chitosan powder. With a syringe, 3.125 mL of the chitosan/HCl solution is transferred to a glass vial. This solution is partly neutralized by adding drop-by-drop 1.875 mL of 0.27 M dibasic sodium phosphate. During the addition of the dibasic sodium phosphate, the solution is stirred vigorously to minimize local basification and avoid formation of local precipitates. This mixing is preferably done at room temperature (20° C.) since the solubility of dibasic sodium phosphate is reduced at lower temperatures. The pH of the resulting solution is near 7.0 at room temperature. The solution is then placed in an incubator at 37° C. whereupon it forms a gel within 15 minutes. A rheological measurement of this mixture, demonstrating thermogelling behavior, is presented in FIG. 1.

### Example 2

### Preparation of an Alternative Thermo Sensitive Chitosan Phosphate Gel

A further example of the application of the above formulae is reported herein. Chitosan from Natural Biopolymer having a degree of deacetylation 85% was dissolved in 120 mM HCl to obtain a molar glucosamine concentration of 141 mM (166 mM as total average mean monomer molar concentration or 2.7% w/v). A disodium phosphate solution of 0.815 M Na₂HPO₄ with 0.288 M HCl was prepared. An initial volume of 200 μL of the Na₂HPO₄ solution was added to 2.0 mL of the chitosan solution and placed in a oven at 60° C. to dissolve some precipitates that form during the previous mixing step. The mixture is then cooled down to room temperature. An additional 200 μL of the same phosphate-HCl solution was then added to the mixture. The resulting solution was placed in an oven at 60° C. whereupon a gel is formed after 25 minutes.

### Example 3

### Alternative Compositions for Thermal Gelation of Polyelectrolyte Solutions

The principle of thermal gelation of polyelectrolyte solutions that is revealed in this invention can be applied to obtain several additional compositions that are logical and direct extensions of the chitosan-inorganic phosphate system described above. The use of alternative buffers other than dibasic sodium phosphate and glycerol 2-phosphate and other polyol phosphates described here is clearly possible and simply depends upon their specific pKₐ and variation of pKₐ with temperature (dpKₐ/dT) as long as equation 13 is respected. Particular volumes and concentrations of solutions to be mixed can then be predicated using the modeling approach described in this invention, for example by calculating the change in ionization state induced by a temperature change with equation 47. One general principle outlined by this invention is that the pKₐ of the weak electrolyte should be close to that of the cationic polyelectrolyte and the pKₐ of the weak electrolyte should be relatively insensitive to temperature, compared to that of the cationic polyelectrolyte in order that heat induced neutralization of the cationic polyelectrolyte occurs. In this manner several mixtures of polyelectrolyte and weak electrolyte may be chosen from components such as those in Table 1, but not limited to those of Table 1, in order to achieve thermosensitive gelation.

Examples using anionic polyelectrolytes can also be identified using the principles taught from this invention. The primary difference with anionic polyelectrolytes is that temperature induced dissociation of protons from an anionic polyelectrolyte will increase charge state of an anionic polyelectrolyte rather than reduce it, as in the case of the above described cationic polyelectrolyte. Thus, in order to form thermosensitive gelling systems using anionic polyelectrolytes the criterion expressed in equation 13 should be reversed such that the tendency of the weak electrolyte to dissociate at higher temperatures is greater than that of the anionic polyelectrolyte, thereby creating a net transfer of protons to the anionic polyelectrolyte and neutralizing it at higher temperatures. Of course such a polyelectrolyte will gel only when attractive hydrophobic forces and hydrogen bonds overcome residual repulsive electrostatic forces due to the partial remaining charged state of the polyelectrolyte. One example of such a system, that is an embodiment of our invention, is a phosphate containing polyelectrolyte, such as a polynucleotide (DNA, RNA), in the presence of an amine containing weak electrolyte, such as glucosamine. Heat induced charge transfer from monomeric glucosamine, to the phosphate containing polyelectrolyte and thereby neutralize it, allowing it to establish hydrogen bonding and gel formation.

Yet another embodiment of the invention is the formation of temperature sensitive gels using anionic polyelectrolytes where the anionic polyelectrolyte transfers protons to the weak electrolyte when heated and thereby becomes more highly charged thereby permitting ionic cross-linking with an oppositely charged cationic species in solution at higher temperature. Such a system can be achieved with the commonly used alginate/calcium ionically cross-linked gel. A thermosensitive system could be produced by tailoring the composition of this system such that the alginate passes from a less charged to a more charged (anionic) state upon heating allowing it to form ionic bonds with calcium and thereby a thermosensitive gel. Using the principles of this invention the exact parameters of such a system can be easily identified.

Yet additional examples of thermosensitive polyelectrolyte/buffer systems may be found by implementing temperature-induced changes of ionization state of system components. Here an example is a composition of the anionic polyelectrolyte alginate to which we add calcium carbonate, CaCO₃ and glucosamine in similar amounts. Alginate is first cooled down, calcium carbonate solution is then added following which we add glucosamine solution and heat the mixture. Upon heating glucosamine will dissociate, thereby liberating protons into solution, decreasing the pH and permit the solubilization of calcium carbonate, since calcium carbonate dissolves easily under acidic pH and higher temperature. Once Ca²⁺ ions are released from calcium carbonate they attract polyanionic alginate chains, form ionic bonds and consequently a solid hydrogel.

### Example 4

### Precipitation Induced by Heating in Diluted Chitosan-Dibasic Sodium Phosphate and Chitosan-GP Solutions

This example shows the precipitation induced by heating in diluted chitosan solutions monitored by a decrease in light transmittance in temperature ramp experiments. These experiments reveal the mechanism of the gelification or homogeneous block-precipitation induced by heating that occurs in concentrated solutions.

Chitosan-GP and chitosan-dibasic sodium phosphate mixtures were prepared and heated using the experimental apparatus described above and shown in FIG. 2. Solutions were prepared by mixing equal volumes of a chitosan solution corresponding to 3 mM of glucosamine monomer and of a GP or dibasic sodium phosphate solution. The final concentration of glucosamine was 1.5 mM for all solutions. A chitosan with f_{D}=72% was used and the heating rate was 1° C./minute.

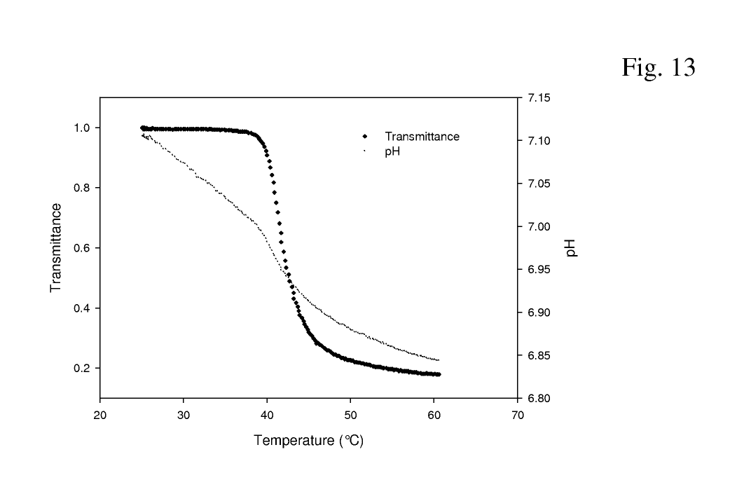

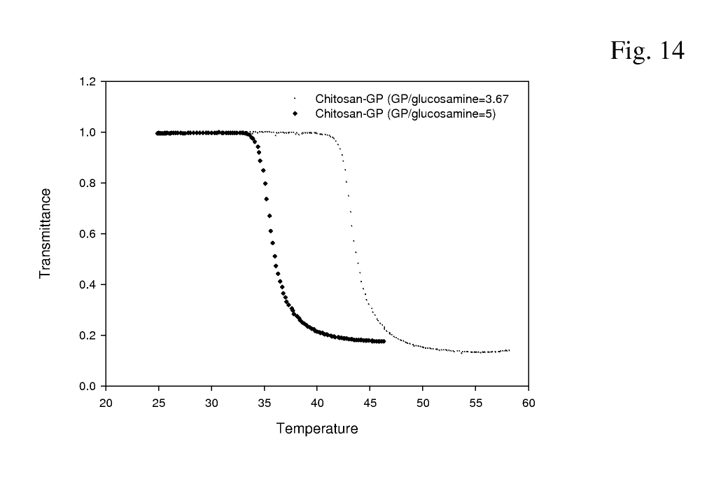

FIG. 13 shows the transmittance and pH of a solution of chitosan-dibasic sodium phosphate (with phosphate/glucosamine molar ratio β of 1.67) as a function of temperature. The precipitation is revealed by a decrease in the transmittance that coincides with a change in the slope of the pH of the solutions.

FIG. 14 shows the transmittance as a function of temperature of two chitosan-GP solutions with GP/glucosamine molar ratios β of 3.67 and 5. The solution of ratio 5 precipitates at a lower temperature since its initial pH is higher and the initial charge state of the polymer is lower. This result is consistent with a transfer of proton induced by heating as the mechanism of gelation.

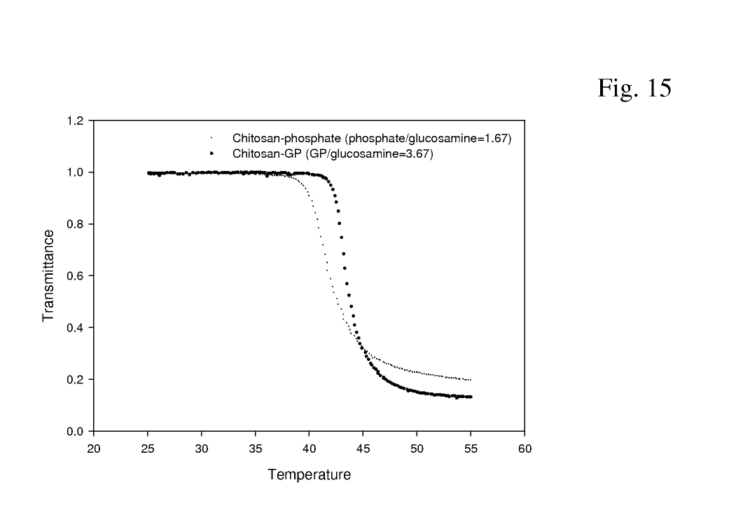

FIG. 15 shows the transmittance as a function of temperature of a solution of chitosan-GP (molar ratio GP/glucosamine=3.67) and a solution of chitosan-disodium phosphate (molar ratio phosphate/glucosamine=1.67). The phosphate solution precipitates at a lower temperature even if the buffer/glucosamine ratio is lower. This can be explained by the higher pKa of phosphate compared to GP that results in a higher initial pH (7.11 vs 7.00 at 25° C.). Both solutions present a similar transmittance decrease during precipitation, however, the precipitation of the chitosan-phosphate solution occurs over a wider range of temperature. This is the result of a smaller variation of the charge state of chitosan with temperature as predicted from equation 11a below considering for the phosphate solution that

$$\frac{d {pK}_{0}^{Glc}}{d T} - \frac{d {pK}_{a 2}^{P}}{d T}$$

and β are both smaller than for the GP solution.

### Example 5

### Precipitation Induced by Heating in a Diluted Chitosan-MES Solution

This example shows the precipitation induced by heating in a diluted chitosan solution monitored by a decrease in light transmittance in a temperature ramp experiment. This experiment reveals the mechanism of the gelation or homogeneous block-precipitation induced by heating that occurs in concentrated solutions. In addition, it shows that the precipitation/gelation for chitosan also occurs with buffers other that phosphate-based buffers.

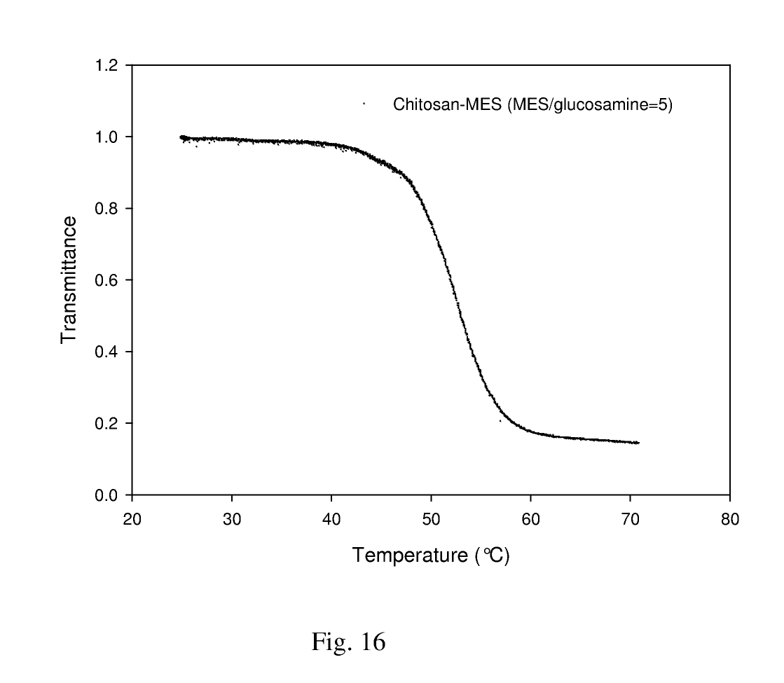

Chitosan-MES (4-Morpholineethanesulfonic acid) solution was heated using the experimental apparatus described above and shown in FIG. 2. The solution was prepared by mixing equal volumes of a chitosan solution corresponding to 3 mM of glucosamine monomer and of a 15 mM MES/15 mM NaOH solution. A chitosan with fD=98% was used and the heating rate was 1° C./minute. FIG. 16 shows the transmittance of the chitosan-MES solution (with MES/glucosamine molar ratio of 5) as a function of temperature. The precipitation is revealed by a decrease in the transmittance.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A thermally sensitive polyelectrolyte composition comprising:
a) a solution of a polyelectrolyte; and
b) a weak electrolyte,
said weak electrolyte being dissolved in the solution of polyelectrolyte and causing said polyelectrolyte to precipitate and form a gel upon heating, when said polyelectrolyte and said weak electrolyte reach a specific charge state,
wherein the polyelectrolyte is a cationic polyelectrolyte,
wherein said specific charge state of the cationic polyelectrolyte for precipitation or gelation is obtained by proton transfer from the polyelectrolyte to the weak electrolyte upon heating said solution, resulting in partial neutralization of the polyelectrolyte, and
wherein the proton transfer occurs when the following formula is satisfied:
$$\frac{d {pK}_{a}^{poly}}{d T} < \frac{d {pK}_{a}^{ion}}{d T}$$
wherein pKₐ^{poly} is the equilibrium dissociation constant of the cationic polyelectrolyte, pKₐ^{ion} is the equilibrium dissociation constant of the weak electrolyte, d is the derivative operator and T the temperature; and
wherein the pKₐ of the weak electrolyte is close to that of the cationic polylectrolyte and the pKₐ of the weak electrolyte is relatively insensitive to temperature, compared to that of the cationic polyelectrolyte in order that heat induced neutralization of the cationic polyelectrolyte occurs.

2. A thermally sensitive polyelectrolyte composition comprising:
a) a solution of a polyelectrolyte; and
b) a weak electrolyte,
said weak electrolyte being dissolved in the solution of polyelectrolyte and causing said polyelectrolyte to precipitate and form a gel upon heating, when said polyelectrolyte and said weak electrolyte reach a specific charge state,
wherein the polyelectrolyte is an anionic polyelectrolyte.
wherein said specific charge state is obtained when there is protonation and neutralization of the anionic polyelectrolyte upon heating said solution; and
wherein the proton transfer occurs when the following formula is satisfied:
$$\frac{d {pK}_{a}^{poly}}{d T} > \frac{d {pK}_{a}^{ion}}{d T}$$
wherein pKₐ^{poly} is the equilibrium dissociation constant of the anionic polyelectrolyte, pKₐ^{ion} is the equilibrium dissociation constant of the weak electrolyte, d is the derivative operator and T the temperature.

3. The composition of claim 2, wherein said specific charge state is obtained when there is deprotonation and ionization of the anionic polyelectrolyte upon heating said solution thereby permitting ionic cross-linking via a small multivalent ion.

4. The composition of any one of claim 1, wherein the weak electrolyte undergoes a change in ionization state causing a change in pH of the solution, thereby inducing polyelectrolyte gel formation.

5. The composition of claim 4, where the weak electrolyte is a weak acid that acidifies the solution upon heating.

6. The composition of claim 4, where the weak electrolyte is a weak base acid that basifies the solution upon heating.

7. The composition of claim 1, wherein the cationic polyelectrolyte is chitosan.

8. The composition of claim 1, wherein the weak electrolyte is selected from the group consisting of dibasic sodium phosphate salt, a phosphate, a phosphonate and MES (4-Morpholineethanesulfonic) acid solution.

9. The composition of any one of claim 1, wherein the polyelectrolyte is linked to the weak electrolyte to constitute a single component auto-gelling system.

10. The composition of claim 8, wherein the polyelectrolyte is linked with a spacer to the weak electrolyte.

11. The composition of claim 9, wherein the polyelectrolyte is covalently linked to the weak electrolyte.

12. The composition of claim 10, wherein the electrolyte is covalently linked with a spacer to the weak electrolyte.

13. The composition of claim 11, wherein the spacer is a saturated or unsaturated branched or unbranched carbon.
